(19) United States
(12) Patent Application Publication
(10) Pub. No.: US 2016/0346433 A1
(43) Pub. Date: Dec. 1, 2016

(54) HYALURONIC ACID COMPOSITIONS INCLUDING MEPIVACAINE

(71) Applicant: LABORATOIRES VIVACY, Archamps (FR)

(72) Inventors: Jérémie BON BETEMPS, Albens (FR); Guy VITALLY, Le Bourget du Lac (FR)

(73) Assignee: LABORATOIRES VIVACY, Archamps (FR)

(21) Appl. No.: 15/138,773

(22) Filed: Apr. 26, 2016

Related U.S. Application Data
(63) Continuation of application No. PCT/EP2014/079270, filed on Dec. 23, 2014.

(30) Foreign Application Priority Data
Dec. 23, 2013 (FR) 13/63505

(51) Int. Cl. A61L 27/54; A61L 27/52; A61L 27/20

(52) U.S. Cl. CPC A61L 27/54; A61L 27/20; A61L 27/52; A61L 2400/06; A61L 2300/402

(57) ABSTRACT

A sterilized aqueous composition includes at least one hyaluronic acid or salt thereof and at least mepivacaine or salt thereof. The weight ratio between the concentration of hyaluronic acid [HA] and the concentration of mepivacaine [MEPI]—[HA]/[MEPI]—is no lower than 0.1. Mepivacaine may be used as a substitute for lidocaine in an equivalent amount in order to obtain a hyaluronic acid composition including a local anaesthetic with rheological properties after heat sterilization that are superior to the rheological properties of the same composition of hyaluronic acid except including lidocaine.

### CROSS-REFERENCE TO RELATED APPLICATIONS

This is a Continuation of PCT/EP2014/079270 filed Dec. 23, 2014, which claims the benefit of FR 13/63505 filed Dec. 23, 2013. The disclosure of the prior applications is hereby incorporated by reference herein in its entirety.

### FIELD

The invention relates to the field of biodegradable gels and hydrogels used as biomaterials and more particularly in the medical and esthetic fields.

Among the medical applications, examples that will be mentioned include injections for replacing deficient biological fluids, for example in joints for replacing synovial fluid, injection following surgery to avoid peritoneal adhesions, periurethral injections for treating incontinence and injections following surgery for correcting presbyopia.

Among the esthetic applications, examples that will be mentioned include injections for filling wrinkles, fine lines and skin defects or for increasing volumes, for example of the lips, the cheekbones, etc.

In all these applications, the gels and hydrogels used must have optimized properties in terms of in vivo persistence, rheology and viscosity to ensure good injectability, these hydrogels being injected using needles that must remain as fine as possible, to ensure the precision of the practitioners' gestures and to minimize the post-injection reactions.

The gels and hydrogels used are based on polymers that are chosen from polysaccharides such as hyaluronic acid, keratan, heparin, cellulose and cellulose derivatives, alginic acid, xanthan, carrageenan, chitosan, chondroitin and biologically acceptable salts thereof.

To improve these gels and/or hydrogels and/or to impart particular properties on them, a certain number of additives may be added thereto.

One of the main drawbacks of the addition of additives is the potential degradation of the rheological and/or viscoelastic properties of the final gels or of their stability, either directly during the addition or during the sterilization phases, or over time, for example on storage.

### BACKGROUND

The article by Michael H Gold (Clinical Interventions in Aging, 2007, 369-376) rapidly retraces the history of the evolution of dermal filling products. The first compositions developed for this purpose were collagen-based. The products Zyderm® (approved by the FDA in 1981) and Zyplast® (approved by the FDA in 1985) were based on bovine collagen. Thereafter, two similar products, but based on human-based collagen, were developed (CosmoDerm® and CosmoPlast® approved by the FDA in 2003).

At the end of the 1980s, Balazs developed the first dermal filling composition based on hyaluronic acid. Improvements have since been made to increase the stability of compositions based on hyaluronic acid.

As indicated in the article by Gold mentioned above, the collagen-based compositions contained lidocaine to attenuate the pain associated with the injection technique. However, in a first stage, the compositions based on hyaluronic acid did not contain any local anesthetic on account of the stability problems due to the additives, as outlined above.

In recent years, efforts have been made with a view to incorporating a local anesthetic, in particular lidocaine, into gels based on hyaluronic acid while at the same time ensuring a certain level of stability. Puragen™ Plus sold by Mentor Corporation is, according to the article by Gold, the first filling composition based on hyaluronic acid comprising lidocaine. Patent application WO 2005/112 888 in the name of Mentor Corporation published on 1 Dec. 2005 describes a method for preparing injectable hydrogels that may comprise lidocaine.

Numerous patent applications relating to compositions based on hyaluronic acid and comprising lidocaine have been filed by researchers in the field.

Patent application WO 2005/067 994 in the name of Anika Therapeutics published on 28 Jul. 2005 describes in Example 21 compositions based on crosslinked hyaluronic acid gel particles comprising lidocaine. Lidocaine is the only exemplified local anesthetic.

Patent application WO 2010/015 901 in the name of Allergan published on 11 Feb. 2010 describes injectable dermal filling compositions based on hyaluronic acid comprising lidocaine. In said document, only compositions based on lidocaine are exemplified.

Patent application WO 2010/052 430 in the name of Anteis published on 14 May 2010 describes compositions based on hyaluronic acid comprising lidocaine and one or more polyol(s).

Patent application WO 2012/104 419 in the name of Q-MED AB published on 9 Aug. 2012 also describes injectable dermal filling compositions based on hyaluronic acid comprising a local anesthetic. In particular, compositions comprising lidocaine, bupivacaine (pKa=8.1) and tetracaine (pKa=8.5) are exemplified. The local anesthetics that are preferred in the description are bupivacaine, lidocaine and ropivacaine (pKa=8.1). No composition incorporating a local anesthetic whose pKa is less than that of lidocaine was thus exemplified or individually cited. Most of the examples described in patent application WO 2012/104 419 relate to compositions comprising lidocaine.

Patent application WO 2013/186 493 discloses hyaluronic acid compositions including a sucrose octasulfate. No formulation comprising a local anesthetic is exemplified and all the examples illustrate compositions that undergo a final sterilization by autoclaving.

Patent application FR 2 979 539 in the name of Teoxane describes formulations comprising a local anesthetic and other active agents; in this case also, only compositions comprising lidocaine are described.

Patent application CN 102805882 relates to hyaluronic acid compositions to which are added local anesthetics, just before their use, but no example is described.

Patent application WO 2013/186 493 discloses hyaluronic acid compositions including a vitamin C derivative. No formulation comprising a local anesthetic is exemplified.

Patent application KR 20140025117 describes compositions based on hyaluronic acid comprising anesthetics, and only compositions comprising lidocaine are described.

Other local anesthetics are mentioned in the literature, but are rarely exemplified, and only products comprising lidocaine are marketed at the present time.

A patent application EP 2 581 079 in the name of Biopolymer GmbH & Co. KG describes compositions based on hyaluronic acid and prilocaine, which has a rapid prilocaine release profile.

### SUMMARY

Indeed, one of the improvements remaining to be achieved is that of obtaining the fastest possible action of the local anesthetics.

Despite all the existing prior art relating to hyaluronic acid compositions comprising a local anesthetic, almost all of the prior art examples relate to lidocaine and no prior art example relates to mepivacaine.

Among the potential candidates are local anesthetics of fast-acting amino amide type, this group being constituted of lidocaine, etidocaine, mepivacaine, prilocaine and articaine.

The action delay of these local anesthetics depends on their pKa, which is between 7.7 and 8.0. At physiological pH, the local anesthetic having the shortest action delay is that whose pKa is the closest to 7.4, since its liposoluble, non-ionized basic form will be that which will penetrate the epinerium and the neuronal membrane, thereafter allowing the molecule to be more rapidly available for blocking the sodium channels. Among the possible candidates of fast-acting amino amide type, mepivacaine is the local anesthetic that has the lowest pKa of the group, since its pKa is 7.7; mepivacaine thus has, in theory, the shortest action delay of the group.

However, one of the risks presented by incorporating molecules of this type is their tendency toward precipitation. Indeed, the basic form is liposoluble, and thus, during their incorporation into the aqueous gel, which is generally formulated at a pH close to physiological pH, i.e. 7.4, the anesthetic will have a high propensity to precipitate.

The precipitation of local anesthetics is quite difficult to apprehend. Usually, the pKa is considered as being a good indicator of precipitation: the lower the pKa, the greater the risks of precipitation (all conditions being otherwise equal). Thus, mepivacaine is, in view of its pKa of 7.7, the worst candidate of the group of local anesthetics of the fast-acting amino amide group from the point of view of the pKa. It is undoubtedly for this reason that mepivacaine has never been exemplified in the prior art. Indeed, even in applications investigating the possibilities of incorporating local anesthetics alternative to lidocaine, for instance patent application WO 2012/104 419 in the name of Q-MED AB, mepivacaine is cited, but only local anesthetics alternative to lidocaine which have a higher pKa are exemplified: bupivacaine (pKa 8.1), tetracaine (pKa 8.5). Finally, prilocaine, which is envisaged in patent application EP 2 581 079 in the name of Biopolymer GmbH & Co. KG, has a pKa of 7.9.

Indeed, it is imperative that no precipitation should take place in gels that are injected with fine needles for the purpose of correcting wrinkles. The use in esthetics demands that nothing should impede the injection, to avoid poor application and thus defective filling. Furthermore, a precipitate would cause the same effects as a foreign body and would thus lead to risks of inflammation. Moreover, the formation of a precipitate would reduce the amount of local anesthetic in solution and would consequently reduce its bioavailability and thus its efficacy.

On account, undoubtedly, of the abovementioned drawbacks, although listed among the local anesthetics that may be incorporated into compositions based on hyaluronic acid especially in patent applications WO 2010/015 901 and WO 2012/104 419, no example of a gel based on hyaluronic acid comprising mepivacaine has been described.

The literature discloses an article by Cho et al., Pak. J. Pharm. Sci., 2001 January; 24(1): 87-93 which describes studies on mepivacaine release from hydroxypropyl methylcellulose (HPMC) gels. These compositions are formulated in gel form for direct application to the skin and transdermal administration. It is described in said article that increasing the concentration of mepivacaine and increasing the temperature increases the rate of release of mepivacaine.

No hyaluronic acid formulation comprising mepivacaine has been described to date, undoubtedly because of the potential difficulties in formulating it at physiological pH.

Surprisingly, the Applicant has shown that the incorporation of mepivacaine into gels based on hyaluronic acid makes it possible firstly to obtain compositions at a pH close to physiological pH without precipitate and despite the unfavorable pKa of mepivacaine, and secondly that these sterilized compositions have less impaired rheological properties during their sterilization than compositions comprising another local anesthetic of the same group.

Furthermore, this reduced impairment of the elastic component G′ during sterilization was observed irrespective of the other possible excipients or additional compounds conventionally used in the formulation of filling gels.

Surprisingly also, the addition of mepivacaine makes it possible to obtain compositions in the presence of polyols which systematically have improved rheological properties when compared with compositions comprising neither polyol nor anesthetic.

The invention thus relates to a sterilized aqueous composition at a pH close to physiological pH, comprising at least one hyaluronic acid and at least mepivacaine, the mass ratio between the concentration of hyaluronic acid [HA] and the concentration of mepivacaine [MEPI]:[HA]/[MEPI] being greater than or equal to 0.1; [HA]/[MEPI]≧0.1.

### BRIEF DESCRIPTION OF THE DRAWING

FIG. 1 is a graph showing the concentrations of lidocaine and mepivacaine as a function of dialysis time in connection with Example 6.

### DETAILED DESCRIPTION OF EMBODIMENTS

The term “hyaluronic acid” means crosslinked or non-crosslinked hyaluronic acid, alone or as a mixture, optionally chemically modified by substitution, alone or as a mixture, optionally in the form of a salt thereof, alone or as a mixture.

The term “mepivacaine” means mepivacaine or a salt thereof, alone or as a mixture.

In one embodiment, the mass ratio between the concentration of hyaluronic acid [HA] and the concentration of mepivacaine [MEPI]:[HA]/[MEPI] is between 0.1 and 50, 0.1 [HA]/[MEPI]≦50.

In one embodiment, the mass ratio between the concentration of hyaluronic acid [HA] and the concentration of mepivacaine [MEPI]:[HA]/[MEPI] is between 0.5 and 40, 0.5≦[HA]/[MEPI]≦40.

In one embodiment, the mass ratio between the concentration of hyaluronic acid [HA] and the concentration of mepivacaine [MEPI]:[HA]/[MEPI] is between 1 and 30, 1≦[HA]/[MEPI]≦30.

In one embodiment, the mass ratio between the concentration of hyaluronic acid [HA] and the concentration of mepivacaine [MEPI]:[HA]/[MEPI] is between 2 and 20, 2≦[HA]/[MEPI]≦20.

In one embodiment, the mass ratio between the concentration of hyaluronic acid [HA] and the concentration of mepivacaine [MEPI], [HA]/[MEPI] is between 7/3 and 26/3, 7/3≦[HA]/[MEPI]≦26/3.

In one embodiment, the mass ratio between the concentration of hyaluronic acid [HA] and the concentration of mepivacaine [MEPI], [HA]/[MEPI] is between 2 and 20/3, 2≦[HA]/[MEPI]≦20/3.

In one embodiment, the mass ratio between the concentration of hyaluronic acid [HA] and the concentration of mepivacaine [MEPI], [HA]/[MEPI] is between 2 and 10/3, 2≦[HA]/[MEPI]≦10/3.

In one embodiment, the mass ratio between the concentration of hyaluronic acid [HA] and the concentration of mepivacaine [MEPI], [HA]/[MEPI] is 20.

In one embodiment, the mass ratio between the concentration of hyaluronic acid [HA] and the concentration of mepivacaine [MEPI], [HA]/[MEPI] is 26/3.

In one embodiment, the mass ratio between the concentration of hyaluronic acid [HA] and the concentration of mepivacaine [MEPI], [HA]/[MEPI] is 20/3.

In one embodiment, the mass ratio between the concentration of hyaluronic acid [HA] and the concentration of mepivacaine [MEPI], [HA]/[MEPI] is 10/3.

In one embodiment, the mass ratio between the concentration of hyaluronic acid [HA] and the concentration of mepivacaine [MEPI], [HA]/[MEPI] is 7/3.

In one embodiment, the mass ratio between the concentration of hyaluronic acid [HA] and the concentration of mepivacaine [MEPI], [HA]/[MEPI] is 2.

In one embodiment, the concentration of mepivacaine [MEPI] is between 0.01 mg/g and 50 mg/g of total weight of said composition.

In one embodiment, the concentration of mepivacaine [MEPI] is between 0.05 mg/g and 45 mg/g of total weight of said composition.

In one embodiment, the concentration of mepivacaine [MEPI] is between 0.1 mg/g and 40 mg/g of total weight of said composition.

In one embodiment, the concentration of mepivacaine [MEPI] is between 0.2 mg/g and 30 mg/g of total weight of said composition.

In one embodiment, the concentration of mepivacaine [MEPI] is between 0.5 mg/g and 20 mg/g of total weight of said composition.

In one embodiment, the concentration of mepivacaine [MEPI] is between 1 mg/g and 15 mg/g of total weight of said composition.

In one embodiment, the concentration of mepivacaine [MEPI] is between 1 mg/g and 10 mg/g of total weight of said composition.

In one embodiment, the concentration of mepivacaine [MEPI] is between 1 mg/g and 6 mg/g of total weight of said composition.

In one embodiment, the concentration of mepivacaine [MEPI] is between 1 mg/g and 5 mg/g of total weight of said composition.

In one embodiment, the concentration of mepivacaine [MEPI] is between 2 mg/g and 5 mg/g of total weight of said composition.

In one embodiment, the concentration of mepivacaine [MEPI] is between 6 mg/g and 10 mg/g of total weight of said composition.

In one embodiment, the concentration of mepivacaine [MEPI] is 1 mg/g of total weight of said composition.

In one embodiment, the concentration of mepivacaine [MEPI] is 3 mg/g of total weight of said composition.

In one embodiment, the concentration of mepivacaine [MEPI] is 4 mg/g of total weight of said composition.

In one embodiment, the concentration of mepivacaine [MEPI] is 5 mg/g of total weight of said composition.

In one embodiment, the concentration of mepivacaine [MEPI] is 6 mg/g of total weight of said composition.

In one embodiment, the concentration of mepivacaine [MEPI] is 10 mg/g of total weight of said composition.

In one embodiment, mepivacaine is chosen from the group comprising mepivacaine or a pharmaceutically acceptable salt thereof.

In one embodiment, mepivacaine is chosen from the group constituted by racemic mepivacaine hydrochloride, racemic mepivacaine, (R)-mepivacaine hydrochloride, (S)-mepivacaine hydrochloride, (R)-mepivacaine and (S)-mepivacaine or a pharmaceutically acceptable salt thereof.

In one embodiment, mepivacaine is racemic mepivacaine hydrochloride.

In one embodiment, mepivacaine is (R)-mepivacaine hydrochloride.

In one embodiment, mepivacaine is (S)-mepivacaine hydrochloride.

In one embodiment, mepivacaine is racemic mepivacaine.

In one embodiment, mepivacaine is (R)-mepivacaine.

In one embodiment, mepivacaine is (S)-mepivacaine.

In one embodiment, the concentration of hyaluronic acid [HA] is between 2 mg/g and 50 mg/g of total weight of said composition.

In one embodiment, the concentration of hyaluronic acid [HA] is between 4 mg/g and 40 mg/g of total weight of said composition.

In one embodiment, the concentration of hyaluronic acid [HA] is between 5 mg/g and 30 mg/g of total weight of said composition.

In one embodiment, the concentration of hyaluronic acid [HA] is between 10 mg/g and 30 mg/g of total weight of said composition.

In one embodiment, the concentration of hyaluronic acid [HA] is 20 mg/g of total weight of said composition.

In one embodiment, the sterilized aqueous composition according to the invention is characterized in that the total content of hyaluronic acid is between 0.2% and 5% by weight relative to the total weight of said composition.

In one embodiment, the sterilized aqueous composition according to the invention is characterized in that the total content of hyaluronic acid is greater than or equal to 1% by weight relative to the total weight of said composition.

In one embodiment, the sterilized aqueous composition according to the invention comprises at least one non-crosslinked hyaluronic acid or a salt thereof, alone or as a mixture.

In one embodiment, the sterilized aqueous composition according to the invention comprises at least one crosslinked hyaluronic acid or a salt thereof, alone or as a mixture.

In one embodiment, the sterilized aqueous composition according to the invention comprises at least one co-crosslinked hyaluronic acid or a salt thereof, alone or as a mixture.

In one embodiment, the sterilized aqueous composition according to the invention comprises at least one crosslinked or non-crosslinked hyaluronic acid chemically modified by substitution, or a salt thereof, alone or as a mixture.

In one embodiment, the hyaluronic acid is doubly crosslinked as described in patent application WO 2000/046 253 in the name of Fermentech Medical Limited.

In one embodiment, the sterilized aqueous composition according to the invention comprises a mixture of crosslinked and non-crosslinked hyaluronic acids, or a salt thereof.

In one embodiment, the sterilized aqueous composition according to the invention comprises a mixture of crosslinked hyaluronic acids, or a salt thereof.

In one embodiment, the mixture of crosslinked hyaluronic acids, or a salt thereof, is a one-phase mixture such as that described in patent application WO 2009/071 697 in the name of the Applicant.

In one embodiment, the mixture of crosslinked hyaluronic acids, or a salt thereof, is a mixture obtained by mixing several hyaluronic acids, or a salt thereof, of different molecular masses prior to their crosslinking, as described in patent application WO 2004/092 222 in the name of Cornéal Industrie.

In one embodiment, the sterilized aqueous composition according to the invention comprises at least one hyaluronic acid, or a salt thereof, substituted with a group providing lipophilic or hydrating properties, for instance the substituted hyaluronic acids as described in patent application FR 2 983 483 in the name of the Applicant.

In one embodiment, the hyaluronic acid is in the sodium salt or potassium salt form.

The term Mw or “molecular mass” means the weight-average molecular mass of the polymers, measured in daltons.

In one embodiment, the composition according to the invention is characterized in that the molecular mass Mw of the at least one hyaluronic acid is within a range from 0.01 MDa to 5 MDa.

In one embodiment, the composition according to the invention is characterized in that the molecular mass Mw of the at least one hyaluronic acid is within a range from 0.1 MDa to 3.5 MDa.

In one embodiment, the composition according to the invention is characterized in that the molecular mass Mw of the at least one hyaluronic acid is within a range from 1 MDa to 3 MDa.

In one embodiment, the composition according to the invention is characterized in that the molecular mass Mw of the at least one hyaluronic acid is 1 MDa.

In one embodiment, the composition according to the invention is characterized in that the molecular mass Mw of the at least one hyaluronic acid is 3 MDa.

In the present invention, the degree of crosslinking X is defined as being equal to the ratio:

$$X = \frac{( \begin{matrix}number of moles of crosslinking \\ agent introduced into the reaction medium\end{matrix} )}{( \begin{matrix}number of moles of dissacharide \\ unit introduced into the reaction medium\end{matrix} )}$$

In one embodiment, the crosslinked hyaluronic acid has a degree of crosslinking X of between 0.001 and 0.5.

In one embodiment, the crosslinked hyaluronic acid has a degree of crosslinking X of between 0.01 and 0.4.

In one embodiment, the crosslinked hyaluronic acid has a degree of crosslinking X of between 0.1 and 0.3.

In one embodiment, the crosslinked hyaluronic acid has a degree of crosslinking X of 0.06.

In one embodiment, the crosslinked hyaluronic acid has a degree of crosslinking X of 0.07.

In one embodiment, the crosslinked hyaluronic acid has a degree of crosslinking X of 0.12.

In one embodiment, the sterilized aqueous composition according to the invention further comprises another polysaccharide.

In one embodiment, this other polysaccharide is chosen from the group constituted by cellulose and derivatives thereof and/or alginic acid or a salt thereof.

The aqueous composition is sterilized, i.e. after it has been prepared, it undergoes a sterilization step, said sterilization step being performed with heat, humid heat, gamma (γ) radiation or a beam of accelerated electrons (electron beam).

In one embodiment, the sterilization step is performed by steam autoclaving.

In one embodiment, the sterilization by steam autoclaving is performed at a temperature of from 121 to 134° C., for a time that is adapted to the temperature.

For example, the sterilization by steam autoclaving is performed at a temperature between 127 and 130° C. for a time of between 1 and 20 minutes.

In one embodiment, the sterilization step is performed by irradiation with gamma (γ) radiation.

In one embodiment, the sterilized aqueous composition according to the invention further comprises at least one antioxidant.

The invention thus also relates to a sterilized aqueous composition, comprising at least one hyaluronic acid, at least mepivacaine and at least one antioxidant, the mass ratio between the concentration of hyaluronic acid [HA] and the concentration of mepivacaine [MEPI]:[HA]/[MEPI] being greater than 0.1; 0.1≦[HA]/[MEPI].

In one embodiment, the sterilized aqueous composition according to the invention is characterized in that the at least one antioxidant is chosen from polyols.

In one embodiment, the sterilized aqueous composition according to the invention is characterized in that the polyols are chosen from the group constituted by glycerol, sorbitol, propylene glycol, xylitol, mannitol, erythritol, maltitol and lactitol, alone or as a mixture.

In one embodiment, the sterilized aqueous composition according to the invention is characterized in that the polyols are chosen from the group constituted by mannitol, sorbitol, maltitol and glycerol, alone or as a mixture.

In one embodiment, the sterilized aqueous composition according to the invention is characterized in that the polyols are chosen from the group constituted by mannitol and sorbitol, alone or as a mixture.

In one embodiment, the sterilized aqueous composition according to the invention is characterized in that the polyol is mannitol.

In one embodiment, the sterilized aqueous composition according to the invention is characterized in that the polyol is sorbitol.

In one embodiment, the sterilized aqueous composition according to the invention is characterized in that the polyol is maltitol.

In one embodiment, the sterilized aqueous composition according to the invention is characterized in that the polyol is glycerol.

In one embodiment, the sterilized aqueous composition according to the invention is characterized in that the antioxidant is a mixture of mannitol and sorbitol.

In general, mannitol, and similarly sorbitol, alone or as a mixture:

afford good resistance to degradation by steam sterilization;

have a high antioxidant power;

are readily dissolved in hyaluronic acid compositions.

In one embodiment, the sterilized aqueous composition according to the invention is characterized in that the polyol content is between 10 and 40 mg/g by total weight of said composition.

In one embodiment, the sterilized aqueous composition according to the invention is characterized in that the polyol content is between 15 and 30 mg/g by total weight of said composition.

In one embodiment, the sterilized aqueous composition according to the invention is characterized in that the polyol content is between 15 and 25 mg/g by total weight of said composition.

In one embodiment, the sterilized aqueous composition according to the invention is characterized in that the polyol content is between 20 and 40 mg/g by total weight of said composition.

In one embodiment, the sterilized aqueous composition according to the invention is characterized in that the polyol content is between 20 and 30 mg/g by total weight of said composition.

In one embodiment, the sterilized aqueous composition according to the invention is characterized in that the polyol content is between 25 and 35 mg/g by total weight of said composition.

In one embodiment, the sterilized aqueous composition according to the invention is characterized in that the polyol content is 35 mg/g by total weight of said composition.

In one embodiment, the sterilized aqueous composition according to the invention is characterized in that the polyol is mannitol and its content is between 10 and 40 mg/g by total weight of said composition.

In one embodiment, the sterilized aqueous composition according to the invention is characterized in that the polyol is mannitol and its content is between 15 and 30 mg/g by total weight of said composition.

In one embodiment, the sterilized aqueous composition according to the invention is characterized in that the polyol is mannitol and its content is between 15 and 25 mg/g by total weight of said composition.

In one embodiment, the sterilized aqueous composition according to the invention is characterized in that the polyol is mannitol and its content is between 20 and 40 mg/g by total weight of said composition.

In one embodiment, the sterilized aqueous composition according to the invention is characterized in that the polyol is mannitol and its content is between 25 and 35 mg/g by total weight of said composition.

In one embodiment, the sterilized aqueous composition according to the invention is characterized in that the polyol is mannitol and its content is 35 mg/g by total weight of said composition.

In one embodiment, the sterilized aqueous composition according to the invention is characterized in that the polyol is sorbitol and its content is between 10 and 40 mg/g by total weight of said composition.

In one embodiment, the sterilized aqueous composition according to the invention is characterized in that the polyol is sorbitol and its content is between 15 and 30 mg/g by total weight of said composition.

In one embodiment, the sterilized aqueous composition according to the invention is characterized in that the polyol is sorbitol and its content is between 15 and 25 mg/g by total weight of said composition.

In one embodiment, the sterilized aqueous composition according to the invention is characterized in that the polyol is sorbitol and its content is between 20 and 40 mg/g by total weight of said composition.

In one embodiment, the sterilized aqueous composition according to the invention is characterized in that the polyol is sorbitol and its content is between 25 and 35 mg/g by total weight of said composition.

In one embodiment, the sterilized aqueous composition according to the invention is characterized in that the polyol is sorbitol and its content is 35 mg/g by total weight of said composition.

In one embodiment, the sterilized aqueous composition according to the invention is characterized in that the polyol is maltitol and its content is between 10 and 40 mg/g by total weight of said composition.

In one embodiment, the sterilized aqueous composition according to the invention is characterized in that the polyol is maltitol and its content is between 15 and 30 mg/g by total weight of said composition.

In one embodiment, the sterilized aqueous composition according to the invention is characterized in that the polyol is maltitol and its content is between 15 and 25 mg/g by total weight of said composition.

In one embodiment, the sterilized aqueous composition according to the invention is characterized in that the polyol is maltitol and its content is between 20 and 40 mg/g by total weight of said composition.

In one embodiment, the sterilized aqueous composition according to the invention is characterized in that the polyol is maltitol and its content is between 25 and 35 mg/g by total weight of said composition.

In one embodiment, the sterilized aqueous composition according to the invention is characterized in that the polyol is maltitol and its content is 35 mg/g by total weight of said composition.

In one embodiment, the sterilized aqueous composition according to the invention is characterized in that the polyol is glycerol and its content is between 10 and 40 mg/g by total weight of said composition.

In one embodiment, the sterilized aqueous composition according to the invention is characterized in that the polyol is glycerol and its content is between 15 and 30 mg/g by total weight of said composition.

In one embodiment, the sterilized aqueous composition according to the invention is characterized in that the polyol is glycerol and its content is between 15 and 25 mg/g by total weight of said composition.

In one embodiment, the sterilized aqueous composition according to the invention is characterized in that the polyol is glycerol and its content is between 20 and 40 mg/g by total weight of said composition.

In one embodiment, the sterilized aqueous composition according to the invention is characterized in that the polyol is glycerol and its content is between 25 and 35 mg/g by total weight of said composition.

In one embodiment, the sterilized aqueous composition according to the invention is characterized in that the polyol is maltitol.

In one embodiment, the sterilized aqueous composition according to the invention is characterized in that the polyol is glycerol and its content is 35 mg/g by total weight of said composition.

In one embodiment, the sterilized aqueous composition according to the invention is characterized in that mepivacaine is freely released in vivo.

In one embodiment, the sterilized aqueous composition according to the invention is characterized in that said composition further comprises at least one additional compound.

In one embodiment, the sterilized aqueous composition according to the invention is characterized in that the content of additional compound is between 0.1 and 100 mg/g of total weight of said composition.

In one embodiment, the sterilized aqueous composition according to the invention is characterized in that the content of additional compound is between 1 and 50 mg/g of total weight of said composition.

In one embodiment, the sterilized aqueous composition according to the invention is characterized in that the additional compound is dimethyl sulfone, referred to hereinafter as DMS.

In one embodiment, the sterilized aqueous composition according to the invention is characterized in that the additional compound is a water-soluble salt of sucrose octasulfate, referred to hereinafter as SOS.

In one embodiment, the sterilized aqueous composition according to the invention is characterized in that the additional compound is a vitamin C derivative.

In one embodiment, the vitamin C derivative is a magnesium ascorbyl phosphate salt, referred to hereinafter as MAP.

In one embodiment, the sterilized aqueous composition according to the invention is characterized in that the additional compound belongs to the catecholamine family.

In one embodiment, the sterilized aqueous composition according to the invention is characterized in that the additional compound belonging to the catecholamine family, is epinephrine.

In one embodiment, the sterilized aqueous composition according to the invention is characterized in that the content of additional compound is between 0.01% and 10% by weight relative to the total weight of said composition.

In one embodiment, the sterilized aqueous composition according to the invention is characterized in that the content of additional compound is between 0.1% and 5% by weight relative to the total weight of said composition.

In one embodiment, the total content of additional compounds is between 0.01 mg/g and 40 mg/g of total weight of said composition.

In one embodiment, the total content of additional compounds is between 0.1 mg/g and 10 mg/g of total weight of said composition.

In one embodiment, the total content of additional compounds is between 0.1 mg/g and 1 mg/g of total weight of said composition.

In one embodiment, the additional compound is dimethyl sulfone and its content is between 1 and 10 mg/g by total weight of said composition.

In one embodiment, the additional compound is a water-soluble salt of sucrose octasulfate and its content is between 1 and 10 mg/g by total weight of said composition.

In one embodiment, the additional compound is a magnesium ascorbyl phosphate salt and its content is between 0.3 and 10 mg/g by total weight of said composition.

The invention also relates to a manufacturing process of a sterilized aqueous composition according to the invention.

In one embodiment, the process according to the invention is characterized in that it comprises at least:
a step of hydration in a buffer solution at a pH close to physiological pH of at least one hyaluronic acid or a salt thereof, alone or as a mixture, to obtain a hydrogel,
a step of incorporating mepivacaine as an aqueous solution in the hydrogel obtained in the preceding step,
a homogenization step, and
a sterilization step.

In one embodiment, the hyaluronic acid is in the form of fibers.

In one embodiment, the hyaluronic acid is in the form of flakes.

In one embodiment, the buffer solution is an aqueous phosphate buffer solution.

In one embodiment, the pH of the mepivacaine solution is adjusted to a value of between 6.5 and 7 before its introduction into the gel and/or hydrogel.

In one embodiment, the pH of the gel and/or hydrogel is adjusted to a value of between 7.7 and 8 before introducing the mepivacaine solution whose pH is not adjusted.

In one embodiment, the mepivacaine solution is incorporated into the gel according to the process described in patent application WO 2010/015 901 in the name of Allergan.

In one embodiment, the process according to the invention is characterized in that the hydration step is performed at room temperature.

In one embodiment, the process according to the invention is characterized in that the homogenization step is performed at room temperature.

In one embodiment, the process according to the invention is characterized in that it further comprises at least one step of packaging the homogenized mixture in syringes.

In one embodiment, the process according to the invention is characterized in that it further comprises at least one step of packaging the homogenized mixture in single-dose flasks.

In one embodiment, the process is characterized in that it comprises at least one sterilization step.

In one embodiment, said sterilization step is performed after the packaging step.

In one embodiment, said sterilization step is performed with heat, humid heat, gamma (γ) radiation, or with a beam of accelerated electrons (electron beam).

In one embodiment, the sterilization step is performed after packaging, by steam autoclaving.

In one embodiment, the sterilization step is performed after packaging, by irradiation with gamma (γ) radiation or with a beam of accelerated electrons (electron beam).

In one embodiment, the process according to the invention is characterized in that the sterilization by steam autoclaving is performed after packaging, at a temperature of from 121 to 134° C., for a time that is adapted to the temperature.

For example, the sterilization by steam autoclaving is performed at a temperature between 127 and 130° C. for a time of between 1 and 20 minutes.

In one embodiment, the process according to the invention is characterized in that it further comprises at least one crosslinking step.

In one embodiment, the process according to the invention is characterized in that the crosslinking step takes place between the hydration step and the step of incorporating mepivacaine.

In one embodiment, the process according to the invention is characterized in that the crosslinking step is performed using at least one crosslinking agent.

In one embodiment, the process according to the invention is characterized in that the crosslinking agent is bifunctional or polyfunctional.

In one embodiment, the process according to the invention is characterized in that the bifunctional or polyfunctional crosslinking agent is chosen from the group constituted by ethylene glycol diglycidyl ether, butanediol diglycidyl ether (BDDE), polyglycerol polyglycidyl ether, polyethylene glycol diglycidyl ether, polypropylene glycol diglycidyl ether, a bis- or polyepoxy such as 1,2,3,4-diepoxybutane or 1,2,7,8-diepoxyoctane, a dialkyl sulfone, divinyl sulfone, formaldehyde, epichlorohydrin or glutaraldehyde, and carbodiimides, for instance 1-ethyl-3-[3-dimethylaminopropyl]carbodiimide hydrochloride (EDC).

In one embodiment, the process according to the invention is characterized in that the bifunctional crosslinking agent is butanediol diglycidyl ether (BDDE) or 1,2,7,8-diepoxyoctane.

In one embodiment, the manufacturing process according to the invention is characterized in that the crosslinking step is performed according to the techniques known to those skilled in the art.

In one embodiment, the process according to the invention is characterized in that it comprises, after the crosslinking step, at least one step of purification and washing performed according to the techniques known to those skilled in the art.

In one embodiment, the process according to the invention is characterized in that it further comprises at least one step of incorporating at least one antioxidant.

In one embodiment, the at least one antioxidant is chosen from polyols.

In one embodiment, the polyols are chosen from the group constituted by glycerol, sorbitol, propylene glycol, xylitol, mannitol, erythritol, maltitol and lactitol, alone or as a mixture.

In one embodiment, the process according to the invention is characterized in that it further comprises at least one step of mixing a solution of at least one additional compound with the hydrogel obtained in the hydration step.

In one embodiment, the process according to the invention is characterized in that the step of mixing a solution of at least one additional compound with the hydrogel obtained in the hydration step is performed before the homogenization step.

In one embodiment, the process according to the invention is characterized in that the step of mixing a solution of at least one additional compound with the hydrogel obtained in the hydration step is performed at a temperature adapted to the manufacturing process. In one embodiment, it is performed at room temperature.

The invention also relates to a process for obtaining a sterilized aqueous composition of hyaluronic acid comprising a local anesthetic, said composition having rheological properties after heat sterilization which are superior to the rheological properties of a composition comprising lidocaine, characterized in that lidocaine is replaced with an equivalent amount, at the same pH, of mepivacaine.

The term “equivalent amount” means either an equivalent amount in mass, in moles or of equivalent bioavailability at a pH close to physiological pH.

In said process, the composition obtained is defined as the composition according to the invention.

The invention also relates to the use of mepivacaine in replacement for lidocaine in equivalent amount to obtain a hyaluronic acid composition comprising a local anesthetic whose rheological properties after heat sterilization are superior to the rheological properties of the same hyaluronic acid composition comprising lidocaine.

In said use, the composition obtained is defined as the composition according to the invention.

The term “equivalent amount” means either an equivalent amount in mass, in moles or of equivalent bioavailability.

The term “hyaluronic acid” means crosslinked or non-crosslinked hyaluronic acid, alone or as a mixture, optionally chemically modified by substitution, alone or as a mixture, optionally in the form of a salt thereof, alone or as a mixture.

The term “local anesthetic” means a local anesthetic or a salt thereof, alone or as a mixture.

The term “mepivacaine” means mepivacaine or a salt thereof, alone or as a mixture.

The term “lidocaine” means lidocaine or a salt thereof, alone or as a mixture.

The term “rheological properties” means the elastic modulus (G′) and/or the viscosity (η).

The term “superior rheological properties” means that the elastic modulus and/or viscosity values are higher.

The term “in replacement” means the formulation of gels in which mepivacaine is incorporated instead of lidocaine.

The invention also relates to the use of mepivacaine for improving the resistance to degradation of the rheological properties of an injectable sterilized aqueous hyaluronic acid composition during heat sterilization.

The invention also relates to the use of mepivacaine for improving the resistance to degradation of the rheological properties of an aqueous hyaluronic acid composition comprising a local anesthetic during heat sterilization.

The invention also relates to the use of mepivacaine in an aqueous hyaluronic acid composition, said composition having degradation of the rheological properties during heat sterilization inferior to that of the same hyaluronic acid composition comprising another local anesthetic.

The invention also relates to a process for improving the resistance to degradation of the rheological properties of an injectable aqueous hyaluronic acid composition during heat sterilization, characterized in that said composition comprises mepivacaine.

The invention also relates to a process for improving the resistance to degradation of the rheological properties of an injectable aqueous hyaluronic acid composition comprising a local anesthetic during heat sterilization, characterized in that said composition comprises mepivacaine.

The invention also relates to a process for improving the resistance to degradation of the rheological properties during heat sterilization of an injectable aqueous hyaluronic acid composition comprising a local anesthetic, by replacing lidocaine with an equivalent amount of mepivacaine.

In one embodiment, the uses or the processes are characterized in that mepivacaine is used in an equivalent amount.

In one embodiment, the uses or the processes are characterized in that the sterilization is performed by steam autoclaving.

In one embodiment, the uses or the processes are characterized in that the other local anesthetic is chosen from the group constituted by lidocaine and prilocaine.

In one embodiment, the uses or the processes are characterized in that the other local anesthetic is lidocaine.

In one embodiment, the uses or the processes are characterized in that the composition further comprises one or more polyol(s).

In one embodiment, the uses or the processes are characterized in that the one or more polyol(s) is(are) chosen from the group constituted by glycerol, sorbitol, propylene glycol, xylitol, mannitol, erythritol, maltitol and lactitol, alone or as a mixture.

In one embodiment, the uses or the processes are characterized in that the one or more polyols are chosen from the group constituted by mannitol, sorbitol, maltitol and glycerol, alone or as a mixture.

In one embodiment, the uses or the processes are characterized in that the one or more polyols are chosen from the group constituted by mannitol and sorbitol, alone or as a mixture.

In one embodiment, the uses or the processes are characterized in that at least one polyol is mannitol.

In one embodiment, the uses or the processes are characterized in that the polyol is mannitol.

In one embodiment, the uses or the processes are characterized in that at least one polyol is sorbitol.

In one embodiment, the uses or the processes are characterized in that the polyol is sorbitol.

In one embodiment, the uses or the processes are characterized in that at least one polyol is maltitol.

In one embodiment, the uses or the processes are characterized in that the polyol is maltitol.

In one embodiment, the uses or the processes are characterized in that at least one polyol is glycerol.

In one embodiment, the uses or the processes are characterized in that the polyol is glycerol.

In one embodiment, the uses or the processes are characterized in that the polyol(s) have a content of between 10 and 40 mg/g by total weight of said composition.

In one embodiment, the uses or the processes are characterized in that the polyol(s) have a content of between 15 and 30 mg/g by total weight of said composition.

In one embodiment, the uses or the processes are characterized in that the polyol(s) have a content of between 15 and 25 mg/g by total weight of said composition.

In one embodiment, the uses or the processes are characterized in that the polyol(s) have a content of between 20 and 40 mg/g by total weight of said composition.

In one embodiment, the uses or the processes are characterized in that the polyol(s) have a content of between 25 and 35 mg/g by total weight of said composition.

In one embodiment, the uses or the processes are characterized in that the polyol(s) have a content of 35 mg/g by total weight of said composition.

In one embodiment, the uses or the processes are characterized in that the polyol is mannitol and its content is between 10 and 40 mg/g by total weight of said composition.

In one embodiment, the uses or the processes are characterized in that the polyol is mannitol and its content is between 15 and 30 mg/g by total weight of said composition.

In one embodiment, the uses or the processes are characterized in that the polyol is mannitol and its content is between 15 and 25 mg/g by total weight of said composition.

In one embodiment, the uses or the processes are characterized in that the polyol is mannitol and its content is between 20 and 40 mg/g by total weight of said composition.

In one embodiment, the uses or the processes are characterized in that the polyol is mannitol and its content is between 25 and 35 mg/g by total weight of said composition.

In one embodiment, the uses or the processes are characterized in that the polyol is mannitol and its content is 35 mg/g by total weight of said composition.

In one embodiment, the uses or the processes are characterized in that the polyol is sorbitol and its content is between 10 and 40 mg/g by total weight of said composition.

In one embodiment, the uses or the processes are characterized in that the polyol is sorbitol and its content is between 15 and 30 mg/g by total weight of said composition.

In one embodiment, the uses or the processes are characterized in that the polyol is sorbitol and its content is between 15 and 25 mg/g by total weight of said composition.

In one embodiment, the uses or the processes are characterized in that the polyol is sorbitol and its content is between 20 and 40 mg/g by total weight of said composition.

In one embodiment, the uses or the processes are characterized in that the polyol is sorbitol and its content is between 25 and 35 mg/g by total weight of said composition.

In one embodiment, the uses or the processes are characterized in that the polyol is sorbitol and its content is 35 mg/g by total weight of said composition.

In one embodiment, the uses or the processes are characterized in that the polyol is maltitol and its content is between 10 and 40 mg/g by total weight of said composition.

In one embodiment, the uses or the processes are characterized in that the polyol is maltitol and its content is between 15 and 30 mg/g by total weight of said composition.

In one embodiment, the uses or the processes are characterized in that the polyol is maltitol and its content is between 10 and 25 mg/g by total weight of said composition.

In one embodiment, the uses or the processes are characterized in that the polyol is maltitol and its content is between 20 and 40 mg/g by total weight of said composition.

In one embodiment, the uses or the processes are characterized in that the polyol is maltitol and its content is between 25 and 35 mg/g by total weight of said composition.

In one embodiment, the uses or the processes are characterized in that the polyol is maltitol and its content is 35 mg/g by total weight of said composition.

In one embodiment, the uses or the processes are characterized in that the polyol is glycerol and its content is between 10 and 40 mg/g by total weight of said composition.

In one embodiment, the uses or the processes are characterized in that the polyol is glycerol and its content is between 15 and 30 mg/g by total weight of said composition.

In one embodiment, the uses or the processes are characterized in that the polyol is glycerol and its content is between 10 and 25 mg/g by total weight of said composition.

In one embodiment, the uses or the processes are characterized in that the polyol is glycerol and its content is between 20 and 40 mg/g by total weight of said composition.

In one embodiment, the uses or the processes are characterized in that the polyol is glycerol and its content is between 25 and 35 mg/g by total weight of said composition.

In one embodiment, the uses or the processes are characterized in that the polyol is glycerol and its content is 35 mg/g by total weight of said composition.

In one embodiment, the uses or the processes are characterized in that the sterilization step is performed by steam autoclaving at a temperature of between 121 to 134° C., for a time adapted to the temperature.

For example, the sterilization by steam autoclaving is performed at a temperature between 127 and 130° C. for a time of between 1 and 20 minutes.

In one embodiment, the uses or the processes are characterized in that the composition further comprises an antioxidant.

In one embodiment, the uses or the processes are characterized in that the mass ratio between the concentration of hyaluronic acid [HA] and the concentration of mepivacaine [MEPI]:[HA]/[MEPI] is greater than or equal to 0.1; [HA]/[MEPI]≧0.1.

In one embodiment, the uses or the processes are characterized in that the mass ratio between the concentration of hyaluronic acid [HA] and the concentration of mepivacaine [MEPI]:[HA]/[MEPI] is between 0.1 and 50, 0.1≦[HA]/[MEPI]≦50.

In one embodiment, the uses or the processes are characterized in that the mass ratio between the concentration of hyaluronic acid [HA] and the concentration of mepivacaine [MEPI]:[HA]/[MEPI] is between 0.5 and 40, 0.5≦[HA]/[MEPI]≦40.

In one embodiment, the uses or the processes are characterized in that the mass ratio between the concentration of hyaluronic acid [HA] and the concentration of mepivacaine [MEPI]:[HA]/[MEPI] is between 1 and 30, 1≦[HA]/[MEPI]≦30.

In one embodiment, the uses or the processes are characterized in that the mass ratio between the concentration of hyaluronic acid [HA] and the concentration of mepivacaine [MEPI]:[HA]/[MEPI] is between 2 and 20, 2≦[HA]/[MEPI]≦20.

In one embodiment, the uses or the processes are characterized in that the mass ratio between the concentration of hyaluronic acid [HA] and the concentration of mepivacaine [MEPI], [HA]/[MEPI] is between 7/3 and 26/3, 7/3≦[HA]/[MEPI]≦26/3.

In one embodiment, the uses or the processes are characterized in that the mass ratio between the concentration of hyaluronic acid [HA] and the concentration of mepivacaine [MEPI], [HA]/[MEPI] is between 2 and 20/3, 2≦[HA]/[MEPI]≦20/3.

In one embodiment, the uses or the processes are characterized in that the mass ratio between the concentration of hyaluronic acid [HA] and the concentration of mepivacaine [MEPI], [HA]/[MEPI] is between 2 and 10/3, 2≦[HA]/[MEPI]≦10/3.

In one embodiment, the uses or the processes are characterized in that the mass ratio between the concentration of hyaluronic acid [HA] and the concentration of mepivacaine [MEPI], [HA]/[MEPI] is 20.

In one embodiment, the uses or the processes are characterized in that the mass ratio between the concentration of hyaluronic acid [HA] and the concentration of mepivacaine [MEPI], [HA]/[MEPI] is 26/3.

In one embodiment, the uses or the processes are characterized in that the mass ratio between the concentration of hyaluronic acid [HA] and the concentration of mepivacaine [MEPI], [HA]/[MEPI] is 20/3.

In one embodiment, the uses or the processes are characterized in that the mass ratio between the concentration of hyaluronic acid [HA] and the concentration of mepivacaine [MEPI], [HA]/[MEPI] is 10/3.

In one embodiment, the uses or the processes are characterized in that the mass ratio between the concentration of hyaluronic acid [HA] and the concentration of mepivacaine [MEPI], [HA]/[MEPI] is 7/3.

In one embodiment, the uses or the processes are characterized in that the mass ratio between the concentration of hyaluronic acid [HA] and the concentration of mepivacaine [MEPI], [HA]/[MEPI] is 2.

In one embodiment, the uses or the processes are characterized in that the concentration of mepivacaine [MEPI] is between 0.01 mg/g and 50 mg/g of total weight of said composition.

In one embodiment, the uses or the processes are characterized in that the concentration of mepivacaine [MEPI] is between 0.01 mg/g and 50 mg/g of total weight of said composition.

In one embodiment, the uses or the processes are characterized in that the concentration of mepivacaine [MEPI] is between 0.05 mg/g and 45 mg/g of total weight of said composition.

In one embodiment, the uses or the processes are characterized in that the concentration of mepivacaine [MEPI] is between 0.1 mg/g and 40 mg/g of total weight of said composition.

In one embodiment, the uses or the processes are characterized in that the concentration of mepivacaine [MEPI] is between 0.2 mg/g and 30 mg/g of total weight of said composition.

In one embodiment, the uses or the processes are characterized in that the concentration of mepivacaine [MEPI] is between 0.5 mg/g and 20 mg/g of total weight of said composition.

In one embodiment, the uses or the processes are characterized in that the concentration of mepivacaine [MEPI] is between 1 mg/g and 15 mg/g of total weight of said composition.

In one embodiment, the uses or the processes are characterized in that the concentration of mepivacaine [MEPI] is between 1 mg/g and 10 mg/g of total weight of said composition.

In one embodiment, the uses or the processes are characterized in that the concentration of mepivacaine [MEPI] is between 1 mg/g and 6 mg/g of total weight of said composition.

In one embodiment, the uses or the processes are characterized in that the concentration of mepivacaine [MEPI] is between 1 mg/g and 5 mg/g of total weight of said composition.

In one embodiment, the uses or the processes are characterized in that the concentration of mepivacaine [MEPI] is between 2 mg/g and 5 mg/g of total weight of said composition.

In one embodiment, the uses or the processes are characterized in that the concentration of mepivacaine [MEPI] is between 6 mg/g and 10 mg/g of total weight of said composition.

In one embodiment, the uses or the processes are characterized in that the concentration of mepivacaine [MEPI] is 1 mg/g of total weight of said composition.

In one embodiment, the uses or the processes are characterized in that the concentration of mepivacaine [MEPI] is 3 mg/g of total weight of said composition.

In one embodiment, the uses or the processes are characterized in that the concentration of mepivacaine [MEPI] is 4 mg/g of total weight of said composition.

In one embodiment, the uses or the processes are characterized in that the concentration of mepivacaine [MEPI] is 5 mg/g of total weight of said composition.

In one embodiment, the uses or the processes are characterized in that the concentration of mepivacaine [MEPI] is 6 mg/g of total weight of said composition.

In one embodiment, the uses or the processes are characterized in that the concentration of mepivacaine [MEPI] is 10 mg/g of total weight of said composition.

In one embodiment, the uses or the processes are characterized in that mepivacaine is chosen from the group comprising mepivacaine or a pharmaceutically acceptable salt thereof.

In one embodiment, the uses or the processes are characterized in that mepivacaine is chosen from the group constituted by racemic mepivacaine hydrochloride, racemic mepivacaine, (R)-mepivacaine hydrochloride, (S)-mepivacaine hydrochloride, (R)-mepivacaine and (S)-mepivacaine or a pharmaceutically acceptable salt thereof.

In one embodiment, the uses or the processes are characterized in that mepivacaine is racemic mepivacaine hydrochloride.

In one embodiment, the uses or the processes are characterized in that mepivacaine is (R)-mepivacaine hydrochloride.

In one embodiment, the uses or the processes are characterized in that mepivacaine is (S)-mepivacaine hydrochloride.

In one embodiment, the uses or the processes are characterized in that mepivacaine is racemic mepivacaine.

In one embodiment, the uses or the processes are characterized in that mepivacaine is (R)-mepivacaine.

In one embodiment, the uses or the processes are characterized in that mepivacaine is (S)-mepivacaine.

In one embodiment, the uses or the processes are characterized in that the concentration of hyaluronic acid [HA] is between 2 mg/g and 50 mg/g of total weight of said composition.

In one embodiment, the uses or the processes are characterized in that the concentration of hyaluronic acid [HA] is between 4 mg/g and 40 mg/g of total weight of said composition.

In one embodiment, the uses or the processes are characterized in that the concentration of hyaluronic acid [HA] is between 5 mg/g and 30 mg/g of total weight of said composition.

In one embodiment, the uses or the processes are characterized in that the concentration of hyaluronic acid [HA] is between 10 mg/g and 30 mg/g of total weight of said composition.

In one embodiment, the uses or the processes are characterized in that the concentration of hyaluronic acid [HA] is 20 mg/g of total weight of said composition.

In one embodiment, the uses or the processes are characterized in that the total content of hyaluronic acid is between 0.2% and 5% by weight relative to the total weight of said composition.

In one embodiment, the uses or the processes are characterized in that the total content of hyaluronic acid is greater than or equal to 1% by weight relative to the total weight of said composition.

In one embodiment, the uses or the processes are characterized in that the sterilized aqueous composition comprises at least one non-crosslinked hyaluronic acid or a salt thereof, alone or as a mixture.

In one embodiment, the uses or the processes are characterized in that the sterilized aqueous composition comprises at least one crosslinked hyaluronic acid or a salt thereof, alone or as a mixture.

In one embodiment, the uses or the processes are characterized in that the sterilized aqueous composition comprises at least one co-crosslinked hyaluronic acid or a salt thereof, alone or as a mixture.

In one embodiment, the uses or the processes are characterized in that the sterilized aqueous composition comprises at least one hyaluronic acid chemically modified by substitution and crosslinked, or a salt thereof, alone or as a mixture.

In one embodiment, the uses or the processes are characterized in that the hyaluronic acid is doubly crosslinked as described in patent application WO 2000/046 253 in the name of Fermentech Medical Limited.

In one embodiment, the uses or the processes are characterized in that the sterilized aqueous composition comprises a mixture of crosslinked hyaluronic acids, or a salt thereof.

In one embodiment, the uses or the processes are characterized in that the mixture of crosslinked hyaluronic acids, or a salt thereof, is a one-phase mixture such as that described in patent application WO 2009/071 697 in the name of the Applicant.

In one embodiment, the uses or the processes are characterized in that the mixture of crosslinked hyaluronic acids, or a salt thereof, is a mixture obtained by mixing several hyaluronic acids, or a salt thereof, of different molecular masses prior to their crosslinking, as described in patent application WO 2004/092 222 in the name of Cornéal Industrie.

In one embodiment, the uses or the processes are characterized in that the sterilized aqueous composition comprises at least one hyaluronic acid, or a salt thereof substituted with a group providing lipophilic or hydrating properties, for instance the substituted hyaluronic acids as described in patent application FR 2 983 483 in the name of the Applicant.

In one embodiment, the uses or the processes are characterized in that the hyaluronic acid is in the sodium salt or the potassium salt form.

In one embodiment, the uses or the processes are characterized in that the molecular mass Mw of the at least one hyaluronic acid is within a range from 0.01 MDa to 5 MDa.

In one embodiment, the uses or the processes are characterized in that the molecular mass Mw of the at least one hyaluronic acid is within a range from 0.1 MDa to 3.5 MDa.

In one embodiment, the uses or the processes are characterized in that the molecular mass Mw of the at least one hyaluronic acid is within a range from 1 MDa to 3 MDa.

In one embodiment, the uses or the processes are characterized in that the molecular mass Mw of the at least one hyaluronic acid is 1 MDa.

In one embodiment, the uses or the processes are characterized in that the molecular mass Mw of the at least one hyaluronic acid is 3 MDa.

In one embodiment, the uses or the processes are characterized in that the crosslinked hyaluronic acid has a degree of crosslinking X of between 0.001 and 0.5.

In one embodiment, the uses or the processes are characterized in that the crosslinked hyaluronic acid has a degree of crosslinking X of between 0.01 and 0.4.

In one embodiment, the uses or the processes are characterized in that the crosslinked hyaluronic acid has a degree of crosslinking X of between 0.1 and 0.3.

In one embodiment, the uses or the processes are characterized in that the crosslinked hyaluronic acid has a degree of crosslinking X of 0.08.

In one embodiment, the uses or the processes are characterized in that the crosslinked hyaluronic acid has a degree of crosslinking X of 0.06.

In one embodiment, the uses or the processes are characterized in that the crosslinked hyaluronic acid has a degree of crosslinking X of 0.12.

In one embodiment, the sterilized aqueous composition according to the invention further comprises another polysaccharide.

In one embodiment, this other polysaccharide is chosen from the group constituted by cellulose and derivatives thereof and/or alginic acid or a salt thereof.

In one embodiment, the uses or the processes are characterized in that the sterilized aqueous composition further comprises another polysaccharide.

In one embodiment, the uses or the processes are characterized in that the sterilized aqueous composition according to the invention further comprises another polysaccharide chosen from the group constituted by cellulose and derivatives thereof and/or alginic acid or a salt thereof.

In one embodiment, the uses or the processes are characterized in that the sterilized aqueous composition further comprises at least one additional compound.

In one embodiment, the uses or the processes are characterized in that the content of additional compound in the composition is between 0.1 and 100 mg/g of total weight of said composition.

In one embodiment, the uses or the processes are characterized in that the content of additional compound in the composition is between 1 and 50 mg/g of total weight of said composition.

In one embodiment, the uses or the processes are characterized in that the additional compound is dimethyl sulfone, referred to hereinafter as DMS.

In one embodiment, the uses or the processes are characterized in that the additional compound is a water-soluble salt of sucrose octasulfate, referred to hereinafter as SOS.

In one embodiment, the uses or the processes are characterized in that the additional compound is a vitamin C derivative.

In one embodiment, the uses or the processes are characterized in that the vitamin C derivative is a magnesium ascorbyl phosphate salt, referred to hereinafter as MAP.

In one embodiment, the uses or the processes are characterized in that the additional compound belongs to the catecholamine family.

In one embodiment, the uses or the processes are characterized in that the additional compound belonging to the catecholamine family, is epinephrine.

In one embodiment, the uses or the processes are characterized in that the content of additional compound is between 0.01% and 10% by weight relative to the total weight of said composition.

In one embodiment, the uses or the processes are characterized in that the content of additional compound is between 0.1% and 5% by weight relative to the total weight of said composition.

In one embodiment, the uses or the processes are characterized in that the total content of additional compounds is between 0.01 mg/g and 40 mg/g of total weight of said composition.

In one embodiment, the uses or the processes are characterized in that the total content of additional compounds is between 0.1 mg/g and 10 mg/g of total weight of said composition.

In one embodiment, the uses or the processes are characterized in that the total content of additional compounds is between 0.1 mg/g and 1 mg/g of total weight of said composition.

In one embodiment, the uses or the processes are characterized in that the additional compound is dimethyl sulfone and its content is between 1 and 10 mg/g by total weight of said composition.

In one embodiment, the uses or the processes are characterized in that the additional compound is a water-soluble salt of sucrose octasulfate and its content is between 1 and 10 mg/g by total weight of said composition.

In one embodiment, the uses or the processes are characterized in that the additional compound is a magnesium ascorbyl phosphate salt and its content is between 0.3 and 10 mg/g by total weight of said composition.

The invention also relates to the use of a sterilized aqueous composition for formulating a composition for filling wrinkles, for correcting skin defects or for volumizing (cheekbones, chin, lips).

The invention also relates to a sterilized aqueous composition according to the invention, for its use for filling wrinkles and/or correcting skin defects.

The invention also relates to the use of a sterilized aqueous composition for formulating a composition that can be injected into a joint as a replacement or supplement for deficient synovial fluid.

The invention also relates to a sterilized aqueous composition according to the invention, for its use as a replacement or supplement for deficient synovial fluid.

The invention also relates to the use of a sterilized aqueous composition according to the invention, for formulating a composition for filling wrinkles.

The invention also relates to the use of a sterilized aqueous composition according to the invention, for formulating a viscosupplementation composition.

The invention also relates to a sterilized aqueous composition according to the invention, for its use as a medicament.

The targeted applications are more particularly applications that are commonly widespread in the field of injectable viscoelastic agents and polysaccharides that are used or that may potentially be used in the following pathologies or treatments:
Esthetic injections into the face: for filling wrinkles, skin defects or volumizing (cheekbones, chin, lips);
Volumizing injections in the body: increasing the breasts and the buttocks, increasing the G spot, vaginal plastic surgery, vaginal lip reconstruction, increasing the size of the penis;
Treatment of arthrosis, injection into the joint as a replacement or supplement for deficient synovial fluid;
Peri-urethral injection for treating urinary incontinence caused by sphincter deficiency;
Post-surgical injection especially for preventing peritoneal adhesions;
Injection following surgery to correct presbyopia by laser scleral incisions;
Injection into the vitreal cavity;
Injection during cataract surgery;
Injection into the genitals.

More particularly, in plastic surgery, depending on its viscoelastic and persistence properties, the sterilized aqueous composition obtained according to the process of the invention may be used:
for filling fine lines or medium-sized or deep wrinkles, and may be injected with needles of fine diameter (for example 27 gauge);
as a volumizer with injection using needles of larger diameter, for example of 22 to 26 gauge, and needles which are longer (for example 30 to 40 mm); in this case, its cohesive nature will make it possible to ensure its maintenance at the site of injection.

The sterilized aqueous composition according to the invention also finds an important application in joint surgery and in dental surgery, for example for filling periodontal pockets.

These examples of use are in no way limiting, the sterilized aqueous composition according to the present invention being more broadly envisaged for:
filling volumes;
generating spaces in certain tissues, thus promoting their optimum functioning;
replacing deficient physiological fluids.

The invention also relates to a kit comprising a sterilized aqueous composition according to the invention, packaged in syringes and sterilized after packaging.

The invention also relates to a kit comprising a sterilized aqueous composition according to the invention, packaged in single-dose flasks and sterilized after packaging.

### EXAMPLES

### a) Manufacture of the Gels

Non-Crosslinked Hyaluronic Acid Gels

Injectable-grade sodium hyaluronate (NaHA) fibers are weighed out in a container. An aqueous phosphate buffer solution is added and the whole is homogenized for about 1 hour with a spatula, at room temperature and at an atmospheric pressure of 900 mmHg.

Crosslinked Hyaluronic Acid Gels

The gels comprising crosslinked hyaluronic acid are obtained according to the procedure described in patent application WO 2009/071 697 in the name of the Applicant, using sodium hyaluronate (NaHA) fibers and butanediol diglycidyl ether (BDDE).

Crosslinked and Interpenetrating Hyaluronic Acid Gels

The gels comprising crosslinked and interpenetrating hyaluronic acid are obtained according to the procedure described in patent application WO 2009/071 697 in the name of the Applicant.

Co-Crosslinked Hyaluronic Acid Gels

The gels comprising co-crosslinked hyaluronic acid are obtained according to the procedure described in patent application WO 86/00079 in the name of Allergan.

Local Anesthetics

The local anesthetics are dissolved in a stabilized phosphate buffer solution at a pH close to physiological pH before their incorporation into the crosslinked or non-crosslinked hyaluronic acid gels.

Antioxidants and Additional Compounds

The antioxidants or the additional compounds are dissolved in a phosphate buffer solution before their incorporation into the crosslinked or non-crosslinked hyaluronic acid gels.

Sterilization

The compositions thus obtained are packaged in syringes which are sterilized by steam autoclaving (T=121° C., 10 min).

### b) Measurements of the Rheological Properties

The elastic components G′ of the compositions comprising crosslinked or non-crosslinked hyaluronic acid before and after sterilization by steam autoclaving were measured using a TA Instruments AR 2000 Ex rheometer, in oscillation at 25° C., the values of the elastic component G′ being recorded at a frequency of 1 Hz.

The viscosity η of the compositions is measured using a TA Instruments AR 2000 Ex rheometer, in controlled stress mode at 25° C. The viscosity value is recorded at a stress of 0.02 s⁻¹.

In the examples that follow, the abbreviations used are as follows: LA: local anesthetic; Aox: antioxidant; AC: additional compound; LIDO: lidocaine; MEPI: mepivacaine; PRILO: prilocaine; HA: hyaluronic acid; % G′: % improvement in the elastic component G′ relative to the reference composition.

### Example 1

Example 1 illustrates the influence of various local anesthetics on the impairment during heat sterilization of the rheological properties of gels of non-crosslinked or crosslinked hyaluronic acid of different molecular masses, at different concentrations and at different degrees of crosslinking.

### Example 1-a

Example 1-a illustrates the influence of various local anesthetics on the impairment during heat sterilization of the rheological properties of a gel of hyaluronic acid with a weight-average molecular mass of 3×10⁶ Da at a concentration of 20 mg/g with a degree of crosslinking X=0.12.

The [HA]/[MEPI] or [HA]/[LA] ratio ranges from 6.67 to 2.

For all the measurements, a reference composition is formulated, by replacing the aqueous solution of local anesthetic with an equivalent amount of aqueous phosphate buffer solution (the additional compounds being conserved).

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.
The percentage loss on sterilization Y or Y′ of the component G′ is calculated as follows:

% loss=G′ before sterilization−G′ after sterilization/G′ before sterilization

TABLE 1

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 1 | none | 0 | none | 0 | none | 0 | n/a | 0 |
| 2 | LIDO | 3 | none | 0 | none | 0 | 6.67 | 32 |
| 3 | LIDO | 6 | none | 0 | none | 0 | 3.33 | 13 |
| 4 | LIDO | 10 | none | 0 | none | 0 | 2 | −4 |
| 5 | MEPI | 3 | none | 0 | none | 0 | 6.67 | 34 |
| 6 | MEPI | 6 | none | 0 | none | 0 | 3.33 | 26 |
| 7 | MEPI | 10 | none | 0 | none | 0 | 2 | 24 |
| 8 | PRILO | 3 | none | 0 | none | 0 | 6.67 | 25 |
| 9 | PRILO | 6 | none | 0 | none | 0 | 3.33 | 2 |

At equivalent concentrations, the rheological properties during heat sterilization of compositions comprising hyaluronic acid with a weight-average molecular mass of 3×10⁶ Da at a concentration of 20 mg/g with a degree of crosslinking X=0.12 are less impaired in the presence of mepivacaine than lidocaine or prilocaine, irrespective of the ratio.

### Example 1-b

Example 1-b illustrates the influence of various anesthetics on the impairment during heat sterilization of the rheological properties of a gel of hyaluronic acid with a weight-average molecular mass of 1×10⁶ Da at a concentration of 20 mg/g with a degree of crosslinking X=0.07.

The [HA]/[MEPI] or [HA]/[LA] ratio ranges from 6.67 to 3.33.

For all the measurements, a reference composition is formulated, by replacing the aqueous solution of local anesthetic with an equivalent amount of aqueous phosphate buffer solution (the additional compounds being conserved).

Table 2 below collates the various compositions tested and the results obtained. The abbreviations used are as follows: LA: local anesthetic; Aox: antioxidant; AC: additional compound; LIDO: lidocaine; MEPI: mepivacaine; PRILO: prilocaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 2

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 10 | none | 0 | none | 0 | none | 0 | n/a | 0 |
| 11 | LIDO | 3 | none | 0 | none | 0 | 6.67 | 37 |
| 12 | LIDO | 6 | none | 0 | none | 0 | 3.33 | 28 |
| 13 | MEPI | 3 | none | 0 | none | 0 | 6.67 | 41 |
| 14 | MEPI | 6 | none | 0 | none | 0 | 3.33 | 40 |
| 15 | PRILO | 3 | none | 0 | none | 0 | 6.67 | 26 |

As in Example 1-a, but in the presence of hyaluronic acid with a lower weight-average molecular mass and at a lower degree of crosslinking, the results obtained are confirmed irrespective of the ratio.

As the best results were obtained in the presence of lidocaine or mepivacaine, the study will be continued by comparing the results obtained only in the presence of mepivacaine or lidocaine.

### Example 1-c

Example 1-c illustrates the influence of various anesthetics on the impairment during heat sterilization of the rheological properties of a gel of hyaluronic acid with a weight-average molecular mass of 3×10⁶ Da at a concentration of 20 mg/g with a degree of crosslinking X=0.06.

The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 3 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % G′: % improvement in the elastic component G′ relative to the reference composition. The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition

TABLE 3

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 16 | none | 0 | none | 0 | none | 0 | n/a | 0 |
| 17 | LIDO | 3 | none | 0 | none | 0 | 6.67 | −1 |
| 18 | MEPI | 3 | none | 0 | none | 0 | 6.67 | 12 |

The results obtained in Example 1-a are confirmed with hyaluronic acid of the same weight-average molecular mass, at the same concentration but with a lower degree of crosslinking. The results obtained in Example 1-b are also confirmed with hyaluronic acid of higher average molecular mass, at the same concentration and at a comparable degree of crosslinking.

### Example 1-d

Example 1-d illustrates the influence of various local anesthetics on the impairment during heat sterilization of the rheological properties of a gel of non-crosslinked hyaluronic acid with a weight-average molecular mass of 3×10⁶ Da at a concentration of 20 mg/g.

The [HA]/[MEPI] or [HA]/[LA] ratio ranges from 20 to 2.

For all the measurements, a reference composition is formulated, by replacing the aqueous solution of local anesthetic with an equivalent amount of aqueous phosphate buffer solution (the additional compounds being conserved).

Table 4 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % η: % improvement in the viscosity η relative to the reference composition.

The percentage improvement in the viscosity η is defined as being:

% improvement η=100*(Z−Z′)/Z

with Z=Percentage loss on sterilization of the viscosity η of the reference composition
and Z′=Percentage loss on sterilization of the viscosity η of the test composition.

TABLE 4

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % η |
| 19 | none | 0 | none | 0 | none | 0 | n/a | 0 |
| 20 | LIDO | 1 | none | 0 | none | 0 | 20 | −29 |
| 21 | LIDO | 3 | none | 0 | none | 0 | 6.67 | −44 |
| 22 | LIDO | 6 | none | 0 | none | 0 | 3.33 | −48 |
| 23 | LIDO | 10 | none | 0 | none | 0 | 2 | −49 |
| 24 | MEPI | 1 | none | 0 | none | 0 | 20 | −21 |
| 25 | MEPI | 3 | none | 0 | none | 0 | 6.67 | −37 |
| 26 | MEPI | 6 | none | 0 | none | 0 | 3.33 | −44 |
| 27 | MEPI | 10 | none | 0 | none | 0 | 2 | −46 |

The results obtained in Examples 1-a and 1-c are confirmed with hyaluronic acid of the same weight-average molecular mass, at the same concentration but non-crosslinked, irrespective of the ratio.

The rheological properties during heat sterilization of compositions comprising hyaluronic acid at a concentration of 20 mg/g are less impaired in the presence of mepivacaine than lidocaine or prilocaine, at equivalent concentrations, irrespective of the ratio, the weight-average molecular mass and the degree of crosslinking.

### Example 2

Example 2 illustrates the influence of various local anesthetics in the presence of an antioxidant on the impairment during heat sterilization of the rheological properties of gels of non-crosslinked or crosslinked hyaluronic acid of different molecular masses, at different concentrations and at different degrees of crosslinking.

### Example 2-a

Example 2-a illustrates the influence of various local anesthetics in the presence of mannitol on the impairment during heat sterilization of the rheological properties of a gel of hyaluronic acid with a weight-average molecular mass of 3×10⁶ Da with a degree of crosslinking X=0.12 at a concentration of 20 mg/g.

The [HA]/[MEPI] or [HA]/[LA] ratio ranges from 20 to 2.

For all the measurements, a reference composition is formulated, by replacing the aqueous solution of local anesthetic with an equivalent amount of aqueous phosphate buffer solution (the additional compounds being conserved).

Table 5 below collates the various compositions tested and the results obtained. The abbreviations used are as follows: LA: local anesthetic; Aox: antioxidant; MAN: mannitol; AC: additional compound; LIDO: lidocaine; MEPI: mepivacaine; PRILO: prilocaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 5

| | LA | | Aox | | AC | | [HA]/[MEPI] | |
|---|---|---|---|---|---|---|---|---|
| | | [LA] | | [Aox] | | [LA] | or | |
| No. | nature | (mg/g) | nature | (mg/g) | nature | (mg/g) | [HA]/[LA] | % G′ |
| 28 | none | 0 | MAN | 35 | none | 0 | n/a | 0 |
| 29 | LIDO | 1 | MAN | 35 | none | 0 | 20 | 11 |
| 30 | LIDO | 3 | MAN | 35 | none | 0 | 6.67 | −36 |
| 31 | LIDO | 6 | MAN | 35 | none | 0 | 3.33 | −85 |
| 32 | LIDO | 10 | MAN | 35 | none | 0 | 2 | −96 |
| 33 | MEPI | 1 | MAN | 35 | none | 0 | 20 | 19 |
| 34 | MEPI | 3 | MAN | 35 | none | 0 | 6.67 | −7 |
| 35 | MEPI | 6 | MAN | 35 | none | 0 | 3.33 | −34 |
| 36 | MEPI | 10 | MAN | 35 | none | 0 | 2 | −50 |
| 37 | PRILO | 3 | MAN | 35 | none | 0 | 6.67 | −60 |
| 38 | PRILO | 6 | MAN | 35 | none | 0 | 3.33 | −92 |

At equivalent concentrations in the presence of mannitol, the rheological properties during heat sterilization of compositions comprising hyaluronic acid with a weight-average molecular mass of 3×10⁶ Da at a concentration of 20 mg/g with a degree of crosslinking X=0.12 are less impaired in the presence of mepivacaine and than lidocaine or prilocaine, irrespective of the ratio.

### Example 2-b

Example 2-b illustrates the influence of various local anesthetics in the presence of an antioxidant on the impairment during heat sterilization of the rheological properties of a gel of hyaluronic acid with a weight-average molecular mass of 1×10⁶ Da at a concentration of 20 mg/g with a degree of crosslinking X=0.07.

The [HA]/[MEPI] or [HA]/[LA] ratio ranges from 6.67 to 3.33.

For all the measurements, a reference composition is formulated, by replacing the aqueous solution of local anesthetic with an equivalent amount of aqueous phosphate buffer solution (the additional compounds being conserved).

Table 6 below collates the various compositions tested and the results obtained. The abbreviations used are as follows: LA: local anesthetic; Aox: antioxidant; MAN: mannitol; AC: additional compound; LIDO: lidocaine; MEPI: mepivacaine; PRILO: prilocaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 6

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 39 | none | 0 | MAN | 35 | none | 0 | n/a | 0 |
| 40 | LIDO | 3 | MAN | 35 | none | 0 | 6.67 | −49 |
| 41 | LIDO | 6 | MAN | 35 | none | 0 | 3.33 | −83 |
| 42 | MEPI | 3 | MAN | 35 | none | 0 | 6.67 | −23 |
| 43 | MEPI | 6 | MAN | 35 | none | 0 | 3.33 | −52 |
| 44 | PRILO | 3 | MAN | 35 | none | 0 | 6.67 | −63 |

As in Example 2-a, but in the presence of hyaluronic acid with a lower weight-average molecular mass and at a lower degree of crosslinking, the results obtained are confirmed irrespective of the ratio.

As the best results were obtained in the presence of lidocaine or mepivacaine, the rest of the study was conducted by comparing only the results obtained in the presence of mepivacaine or lidocaine.

### Example 2-c

Example 2-c illustrates the influence of various local anesthetics in the presence of an antioxidant on the impairment during heat sterilization of the rheological properties of a gel of hyaluronic acid with a weight-average molecular mass of 3×10⁶ Da at a concentration of 20 mg/g with a degree of crosslinking X=0.06.

The [HA]/[MEPI] or [HA]/[LA] ratio is 3.33.

For all the measurements, a reference composition is formulated, by replacing the aqueous solution of local anesthetic with an equivalent amount of aqueous phosphate buffer solution (the additional compounds being conserved).

Table 7 below collates the various compositions tested and the results obtained. The abbreviations used are as follows: LA: local anesthetic; Aox: antioxidant; MAN: mannitol; AC: additional compound; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 7

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 45 | none | 0 | MAN | 35 | none | 0 | n/a | 0 |
| 46 | LIDO | 3 | MAN | 35 | none | 0 | 3.33 | −168 |
| 47 | MEPI | 3 | MAN | 35 | none | 0 | 3.33 | −95 |

The results obtained in Example 2-a are confirmed with hyaluronic acid of the same weight-average molecular mass, at the same concentration but with a lower degree of crosslinking. The results obtained in Example 2-b are also confirmed with hyaluronic acid of higher weight-average molecular mass, at the same concentration and with a similar degree of crosslinking.

### Example 2-D

Example 2-d illustrates the influence of various local anesthetics in the presence of mannitol on the impairment during heat sterilization of the rheological properties of a gel of non-crosslinked hyaluronic acid with a weight-average molecular mass of 3×10⁶ Da at a concentration of 20 mg/g.

The [HA]/[MEPI] or [HA]/[LA] ratio ranges from 20 to 2.

For all the measurements, a reference composition is formulated, by replacing the aqueous solution of local anesthetic with an equivalent amount of aqueous phosphate buffer solution.

Table 8 below collates the various compositions tested and the results obtained. The abbreviations used are as follows: LA: local anesthetic; Aox: antioxidant; MAN: mannitol; AC: additional compound; MEPI: mepivacaine; HA: hyaluronic acid; % η: % improvement in the viscosity η relative to the reference composition.

The percentage improvement in the viscosity η is defined as being:

% improvement η=100*(Z−Z′)/Z

with Y=Percentage loss on sterilization of the viscosity η of the reference composition
and Y′=Percentage loss on sterilization of the viscosity η of the test composition.

TABLE 8

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % η |
| 48 | none | 0 | MAN | 35 | none | 0 | n/a | 0 |
| 49 | LIDO | 1 | MAN | 35 | none | 0 | 20 | −50 |
| 50 | LIDO | 3 | MAN | 35 | none | 0 | 6.67 | −66 |
| 51 | LIDO | 6 | MAN | 35 | none | 0 | 3.33 | −69 |
| 52 | LIDO | 10 | MAN | 35 | none | 0 | 2 | −70 |
| 53 | MEPI | 1 | MAN | 35 | none | 0 | 20 | −43 |
| 54 | MEPI | 3 | MAN | 35 | none | 0 | 6.67 | −62 |
| 55 | MEPI | 6 | MAN | 35 | none | 0 | 3.33 | −67 |
| 56 | MEPI | 10 | MAN | 35 | none | 0 | 2 | −68 |

The results obtained in Examples 2-a and 2-c are confirmed with hyaluronic acid of the same weight-average molecular mass, at the same concentration but non-crosslinked, irrespective of the ratio.

At equivalent concentrations, the rheological properties during heat sterilization of compositions comprising hyaluronic acid at a concentration of 20 mg/g comprising mannitol are less impaired in the presence of mepivacaine than lidocaine, irrespective of the ratio, the weight-average molecular mass and the degree of crosslinking.

### Example 3

Example 3 illustrates the influence of various local anesthetics in the presence of a magnesium ascorbyl phosphate salt, referred to hereinafter as MAP, on the impairment during heat sterilization of the rheological properties of hyaluronic acid gels at different degrees of crosslinking.

### Example 3-a

Example 3-a illustrates the influence of various local anesthetics in the presence of MAP on the impairment during heat sterilization of the rheological properties of a gel of hyaluronic acid with a weight-average molecular mass of 3×10⁶ Da with a degree of crosslinking X=0.12 at a concentration of 20 mg/g.

The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 9 below collates the various compositions tested and the results obtained. The abbreviations used are as follows: LA: local anesthetic; Aox: antioxidant; AC: additional compound; MAP: magnesium ascorbyl phosphate; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 9

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 57 | none | 0 | none | 0 | MAP | 0.3 | n/a | 0 |
| 58 | LIDO | 3 | none | 0 | MAP | 0.3 | 6.67 | −42 |
| 59 | MEPI | 3 | none | 0 | MAP | 0.3 | 6.67 | −28 |

In the presence of MAP, the rheological properties during heat sterilization of compositions comprising hyaluronic acid with a weight-average molecular mass of 3×10⁶ Da with a degree of crosslinking X=0.06 and at a concentration of 20 mg/g are less impaired in the presence of mepivacaine than lidocaine, at equivalent concentrations, at a ratio [HA]/[MEPI] of 6.67.

### Example 3-b

Example 3-b illustrates the influence of various local anesthetics in the presence of MAP on the impairment during heat sterilization of the rheological properties of gels of hyaluronic acid with a weight-average molecular mass of 3×10⁶ Da with a degree of crosslinking X=0.06 at a concentration of 20 mg/g.

The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 10 below collates the various compositions tested and the results obtained. The abbreviations used are as follows: LA: local anesthetic; Aox: antioxidant; AC: additional compound; MAP: magnesium ascorbyl phosphate; LIDO: lidocaine; MEPI: mepivacaine; HA: hyaluronic acid; % G′; % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 10

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 60 | none | 0 | none | 0 | MAP | 0.3 | n/a | 0 |
| 61 | LIDO | 3 | none | 0 | MAP | 0.3 | 6.67 | −42 |
| 62 | MEPI | 3 | none | 0 | MAP | 0.3 | 6.67 | −29 |

The results obtained in Example 3-a are confirmed with hyaluronic acid of the same weight-average molecular mass, at the same concentration and with the same ratio [HA]/[MEPI], but with a lower degree of crosslinking.

The rheological properties during heat sterilization of compositions comprising hyaluronic acid at a concentration of 20 mg/g in the presence of MAP are less impaired in the presence of mepivacaine than lidocaine, at equivalent concentrations, irrespective of the degree of crosslinking.

### Example 4

Example 4 illustrates the influence of various local anesthetics in the presence of mannitol and SOS on the impairment during heat sterilization of the rheological properties of gels of hyaluronic acid with a weight-average molecular mass of 3×10⁶ Da with a degree of crosslinking X=0.12 at a concentration of 20 mg/g.

The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 11 below collates the various compositions tested and the results obtained. The abbreviations used are as follows: LA: local anesthetic; Aox: antioxidant; MAN: mannitol; AC: additional compound; SOS: sucrose octasulfate; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 11

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 63 | none | 0 | MAN | 35 | SOS | 1 | n/a | 0 |
| 64 | LIDO | 3 | MAN | 35 | SOS | 1 | 6.67 | −21 |
| 65 | MEPI | 3 | MAN | 35 | SOS | 1 | 6.67 | −14 |

At equivalent concentrations, in the presence of mannitol and SOS, the rheological properties during heat sterilization of compositions comprising hyaluronic acid at a concentration of 20 mg/g are less impaired in the presence of mepivacaine than lidocaine, at a ratio of 6.67.

### Example 5

Example 5 illustrates the influence of various local anesthetics in the presence of mannitol and MAP on the impairment during heat sterilization of the rheological properties of gels of hyaluronic acid with a weight-average molecular mass of 3×10⁶ Da with a degree of crosslinking X=0.12 at a concentration of 20 mg/g.

### Example 5-a

Example 5-a illustrates the influence of various local anesthetics in the presence of mannitol and MAP at a concentration of 0.3 mg/g on the impairment during heat sterilization of the rheological properties of gels of hyaluronic acid with a weight-average molecular mass of 3×10⁶ Da with a degree of crosslinking X=0.12 at a concentration of 20 mg/g.

For all the measurements, a reference composition is formulated, by replacing the aqueous solution of local anesthetic with an equivalent amount of aqueous phosphate buffer solution (the additional compounds being conserved).

Table 12 below collates the various compositions tested and the results obtained. The abbreviations used are as follows: LA: local anesthetic; Aox: antioxidant; MAN: mannitol; AC: additional compound; MAP: magnesium ascorbyl phosphate; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 12

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 66 | none | 0 | MAN | 35 | MAP | 0.3 | n/a | 0 |
| 67 | LIDO | 3 | MAN | 35 | MAP | 0.3 | 6.67 | −30 |
| 68 | MEPI | 3 | MAN | 35 | MAP | 0.3 | 6.67 | −20 |

At equivalent concentrations, the rheological properties during heat sterilization of compositions comprising hyaluronic acid at a concentration of 20 mg/g in the presence of mannitol and MAP at a concentration of 0.3 mg/g are less impaired in the presence of mepivacaine than lidocaine, at a ratio of 6.67.

### Example 5-b

Example 5-b illustrates the influence of various local anesthetics in the presence of mannitol and MAP at a concentration of 0.7 mg/g on the impairment during heat sterilization of the rheological properties of gels of hyaluronic acid with a weight-average molecular mass of 3×10⁶ Da with a degree of crosslinking X=0.12 at a concentration of 20 mg/g.

For all the measurements, a reference composition is formulated, by replacing the aqueous solution of local anesthetic with an equivalent amount of aqueous phosphate buffer solution (the additional compounds being conserved).

Table 13 below collates the various compositions tested and the results obtained. The abbreviations used are as follows: LA: local anesthetic; Aox: antioxidant; MAN: mannitol; AC: additional compound; MAP: magnesium ascorbyl phosphate; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 13

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 69 | none | 0 | MAN | 35 | MAP | 0.7 | n/a | 0 |
| 70 | LIDO | 3 | MAN | 35 | MAP | 0.7 | 6.67 | −29 |
| 71 | MEPI | 3 | MAN | 35 | MAP | 0.7 | 6.67 | −17 |

The results obtained in Example 5-a are confirmed with a higher concentration of MAP.

### Example 6

Example 6 makes it possible to compare the kinetics of mepivacaine release with the kinetics of lidocaine release each introduced into a gel of hyaluronic acid with a weight-average molecular mass of 3×10⁶ Da with a degree of crosslinking X=0.12 at a concentration of 20 mg/g. The initial concentrations of mepivacaine or lidocaine are 3 mg/g.

The protocol for studying the release kinetics of the two local anesthetics is the same as that used in Example 5 of patent application WO 2010/015 901 in the name of Allergan. The release kinetics were, however, studied at 37° C. in physiological saline medium. Monitoring by UV-visible spectrophotometry is performed to assay the local anesthetic present in the gel.

In Table 13 below, the mass percentages of lidocaine or mepivacaine in the hyaluronic acid gel measured after various dialysis times are specified.

TABLE 14

| Time (h) | Lidocaine | Mepivacaine |
|---|---|---|
| 0 | 0.293 | 0.290 |
| 1.5 | 0.160 | 0.133 |
| 5 | 0.109 | 0.100 |
| 7 | 0.103 | 0.098 |
| 23 | 0.091 | 0.086 |
| 48 | 0.084 | 0.077 |
| 72 | 0.082 | 0.075 |

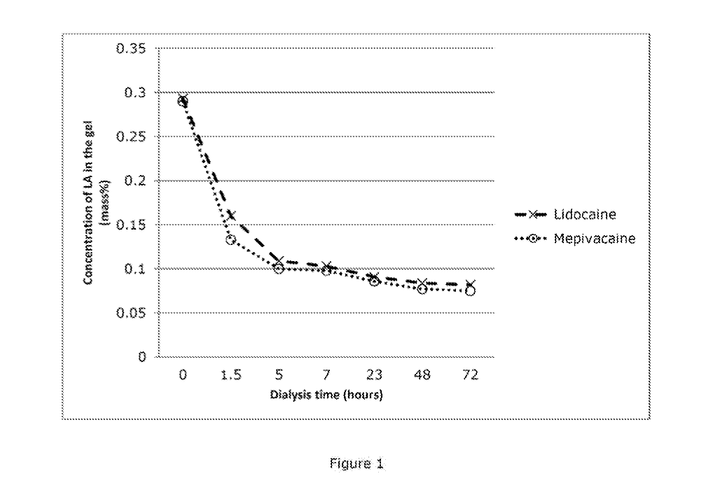

The results obtained above are illustrated by FIG. 1, which is a graph showing the concentrations of lidocaine and mepivacaine as a function of the dialysis time. FIG. 1, which shows the mass concentration of local anesthetic (lidocaine and/or mepivacaine) as a function of the dialysis time in hours, shows that the lidocaine and the mepivacaine release kinetics are comparable.

Thus, the bioavailable amount of local anesthetic is equivalent whether lidocaine is incorporated or mepivacaine is incorporated.

### Example 7

Example 7 illustrates the influence of various local anesthetics on the impairment during heat sterilization of the rheological properties of an interpenetrating gel of hyaluronic acid prepared according to patent application WO 2009/071 697 in the name of Vivacy, and comprising or not comprising mannitol.

### Example 7-a

Example 7-a illustrates the influence of various local anesthetics on the impairment during heat sterilization of the rheological properties of an interpenetrating gel of hyaluronic acid (not comprising mannitol) prepared according to patent application WO 2009/071 697 in the name of Vivacy, at a final concentration of 20 mg/g, the first gel being crosslinked with NaHA with a weight-average molecular mass of 1×10⁶ Da with a degree of crosslinking X=0.03, and the second gel being crosslinked with NaHA with a weight-average molecular mass of 3×10⁶ Da with a degree of crosslinking X=0.06. The mixing ratio of the two gels is 50/50. The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 15 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 15

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 72 | none | 0 | NA | 0 | none | 0 | n/a | 0 |
| 73 | LIDO | 3 | NA | 0 | none | 0 | 6.67 | −9 |
| 74 | MEPI | 3 | NA | 0 | none | 0 | 6.67 | 8 |

At equivalent concentrations, the rheological properties during heat sterilization of compositions comprising crosslinked and interpenetrating hyaluronic acid prepared according to patent application WO 2009/071 697 in the name of Vivacy are less impaired in the presence of mepivacaine than lidocaine.

### Example 7-b

Example 7-b illustrates the influence of various local anesthetics on the impairment during heat sterilization of the rheological properties of an interpenetrating gel of hyaluronic acid comprising mannitol, prepared according to patent application WO 2009/071 697 in the name of Vivacy, at a final concentration of 20 mg/g, the first gel being crosslinked with NaHA with a weight-average molecular mass of 1×10⁶ Da with a degree of crosslinking X=0.03, and the second gel being crosslinked with NaHA with a weight-average molecular mass of 3×10⁶ Da with a degree of crosslinking X=0.06. The mixing ratio of the two gels is 50/50. The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 16 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 16

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 75 | none | 0 | mannitol | 35 | none | 0 | n/a | 0 |
| 76 | LIDO | 3 | mannitol | 35 | none | 0 | 6.67 | −131 |
| 77 | MEPI | 3 | mannitol | 35 | none | 0 | 6.67 | −102 |

At equivalent concentrations, the rheological properties during heat sterilization of compositions comprising crosslinked and interpenetrating hyaluronic acid prepared according to patent application WO 2009/071 697 in the name of Vivacy and mannitol are less impaired in the presence of mepivacaine than lidocaine.

### Example 8

Example 8 illustrates the influence of various local anesthetics (with and without mannitol) on the impairment during heat sterilization of the rheological properties of a gel of co-crosslinked hyaluronic acid prepared according to patent application WO 86/00079 in the name of Allergan.

### Example 8-a

Example 8-a illustrates the influence of various local anesthetics (without mannitol) on the impairment during heat sterilization of the rheological properties of a gel of co-crosslinked hyaluronic acid at a final concentration of 20 mg/g, prepared according to patent application WO 86/00079 in the name of Allergan: during the crosslinking (1% NaOH, degree of crosslinking X=0.09, 50° C.—2 hours 30 min), 2 molecular masses of NaHA are mixed in 50/50 proportion: the first being of a weight-average molecular mass of 1×10⁶ Da, and the second of a weight-average molecular mass of 3×10⁶ Da. The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 17 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 17

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 78 | none | 0 | NA | 0 | none | 0 | n/a | 0 |
| 79 | LIDO | 3 | NA | 0 | none | 0 | 6.67 | 22 |
| 80 | MEPI | 3 | NA | 0 | none | 0 | 6.67 | 26 |

At equivalent concentrations, the rheological properties during heat sterilization of compositions comprising co-crosslinked hyaluronic acid prepared according to patent application WO 86/00079 in the name of Allergan are less impaired in the presence of mepivacaine than lidocaine.

### Example 8-b

Example 8-b illustrates the influence of various local anesthetics (with mannitol) on the impairment during heat sterilization of the rheological properties of a gel of co-crosslinked hyaluronic acid at a final concentration of 20 mg/g, prepared according to patent application WO 86/00079 in the name of Allergan: during the crosslinking (1% NaOH, degree of crosslinking X=0.09, 50° C.−2 hours 30 min), 2 molecular masses of NaHA are mixed in 50/50 proportion: the first being of a weight-average molecular mass of 1×10⁶ Da, and the second of a weight-average molecular mass of 3×10⁶ Da. The [HA]/[MEPI] or [HA]/[LA] ratio is 6.67.

For all the measurements, a reference composition is formulated, by replacing the aqueous solution of local anesthetic with an equivalent amount of aqueous phosphate buffer solution (the additional compounds being conserved).

Table 18 below collates the various compositions tested and the results obtained. The abbreviations used are as follows: LA: local anesthetic; Aox: antioxidant; AC: additional compound; LIDO: lidocaine; MEPI: mepivacaine; HA: hyaluronic acid; % G′: % improvement in the elastic component G′ relative to the reference composition.

The percentage improvement in the elastic component G′ is defined as being:

% improvement G′=100*(Y−Y′)/Y

with Y=Percentage loss on sterilization of the elastic component G′ of the reference composition
and Y′=Percentage loss on sterilization of the elastic component G′ of the test composition.

TABLE 18

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 81 | none | 0 | mannitol | 35 | none | 0 | n/a | 0 |
| 82 | LIDO | 3 | mannitol | 35 | none | 0 | 6.67 | −32 |
| 83 | MEPI | 3 | mannitol | 35 | none | 0 | 6.67 | −22 |

At equivalent concentrations, the rheological properties during heat sterilization of compositions comprising co-crosslinked hyaluronic acid prepared according to patent application WO 86/00079 in the name of Allergan and mannitol are less impaired in the presence of mepivacaine than lidocaine.

### Example 9

Example 9 illustrates the influence of adding various anesthetics on the impairment during heat sterilization of the rheological properties of various hyaluronic acid gels comprising various polyols.

### Example 9-a

Example 9-a consists of a compilation of tests relating to hyaluronic acid with a weight-average molecular mass of 3×10⁶ Da with a degree of crosslinking X=0.06 at a concentration of 20 mg/g.

All the calculations of % G′ are performed taking as reference the composition comprising neither polyol nor local anesthetic nor additional compound.

TABLE 19

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 16 | none | 0 | none | 0 | none | 0 | n/a | 0 |
| 45 | none | 0 | mannitol | 35 | none | 0 | n/a | 54 |
| 17 | LIDO | 3 | none | 0 | none | 0 | 6.67 | −1 |
| 18 | MEPI | 3 | none | 0 | none | 0 | 6.67 | 12 |
| 46 | LIDO | 3 | mannitol | 35 | none | 0 | 6.67 | −23 |
| 47 | MEPI | 3 | mannitol | 35 | none | 0 | 6.67 | 11 |

The rheological properties during heat sterilization of compositions comprising hyaluronic acid with a weight-average molecular mass of 3×10⁶ Da at a concentration of 20 mg/g with a degree of crosslinking X=0.06 are less impaired in the presence of mepivacaine, relative to the reference composition.

### Example 9-b

Example 9-b consists of a compilation of tests relating to hyaluronic acid with a weight-average molecular mass of 3.2×10⁶ Da with a degree of crosslinking X=0.06 at a concentration of 20 mg/g.

All the calculations of % G′ are performed taking as reference the composition comprising neither polyol nor local anesthetic nor additional compound.

TABLE 20

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 84 | none | 0 | none | 0 | none | 0 | n/a | 0 |
| 96 | none | 0 | mannitol | 35 | none | 0 | n/a | 56 |
| 90 | none | 0 | glycerol | 35 | none | 0 | n/a | 54 |
| 87 | none | 0 | sorbitol | 35 | none | 0 | n/a | 46 |
| 93 | none | 0 | maltitol | 35 | none | 0 | n/a | 51 |
| 85 | LIDO | 3 | None | 0 | none | 0 | 6.67 | −9 |
| 86 | MEPI | 3 | None | 0 | none | 0 | 6.67 | 5 |
| 97 | LIDO | 3 | mannitol | 35 | none | 0 | 6.67 | −1 |
| 91 | LIDO | 3 | glycerol | 35 | none | 0 | 6.67 | −2 |
| 88 | LIDO | 3 | sorbitol | 35 | none | 0 | 6.67 | −7 |
| 94 | LIDO | 3 | maltitol | 35 | none | 0 | 6.67 | −2 |
| 98 | MEPI | 3 | mannitol | 35 | none | 0 | 6.67 | 7 |
| 92 | MEPI | 3 | glycerol | 35 | none | 0 | 6.67 | 7 |
| 89 | MEPI | 3 | sorbitol | 35 | none | 0 | 6.67 | 8 |
| 95 | MEPI | 3 | maltitol | 35 | none | 0 | 6.67 | 12 |

The rheological properties during heat sterilization of compositions comprising hyaluronic acid with a weight-average molecular mass of 3.2×10⁶ Da at a concentration of 20 mg/g with a degree of crosslinking X=0.06 are less impaired in the presence of mepivacaine, relative to the reference composition, irrespective of the polyol incorporated into the composition.

The composition having the best results is a composition comprising maltitol and mepivacaine.

### Example 9-c

Example 9-c consists of a compilation of tests relating to hyaluronic acid with a weight-average molecular mass of 1×10⁶ Da with a degree of crosslinking X=0.07 at a concentration of 20 mg/g.

All the calculations of % G′ are performed taking as reference the composition comprising neither polyol nor local anesthetic nor additional compound.

TABLE 21

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 10 | none | 0 | none | 0 | none | 0 | n/a | 0 |
| 39 | none | 0 | mannitol | 35 | none | 0 | n/a | 42 |
| 11 | LIDO | 3 | none | 0 | none | 0 | 6.67 | 37 |
| 13 | MEPI | 3 | none | 0 | none | 0 | 6.67 | 41 |
| 40 | LIDO | 3 | mannitol | 35 | none | 0 | 6.67 | 14 |
| 42 | MEPI | 3 | mannitol | 35 | none | 0 | 6.67 | 29 |

The rheological properties during heat sterilization of compositions comprising hyaluronic acid with a weight-average molecular mass of 1×10⁶ Da at a concentration of 20 mg/g with a degree of crosslinking X=0.07 are less impaired in the presence of mepivacaine, relative to the reference composition.

### Example 9-d

Example 9-d consists of a compilation of tests relating to hyaluronic acid with a weight-average molecular mass of 3×10⁶ Da with a degree of crosslinking X=0.12 at a concentration of 20 mg/g.

All the calculations of % G′ are performed taking as reference the composition comprising neither polyol nor local anesthetic nor additional compound.

TABLE 22

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 1 | none | 0 | none | 0 | none | 0 | n/a | 0 |
| 28 | none | 0 | mannitol | 35 | none | 0 | n/a | 39 |
| 2 | LIDO | 3 | none | 0 | none | 0 | 6.67 | 32 |
| 5 | MEPI | 3 | none | 0 | none | 0 | 6.67 | 34 |
| 30 | LIDO | 3 | mannitol | 35 | none | 0 | 6.67 | 17 |
| 34 | MEPI | 3 | mannitol | 35 | none | 0 | 6.67 | 35 |
| 57 | none | 0 | none | 0 | MAP | 0.3 | n/a | 35 |
| 58 | LIDO | 3 | none | 0 | MAP | 0.3 | 6.67 | 8 |
| 59 | MEPI | 3 | none | 0 | MAP | 0.3 | 6.67 | 17 |
| 66 | none | 0 | mannitol | 35 | MAP | 0.3 | n/a | 39 |
| 67 | LIDO | 3 | mannitol | 35 | MAP | 0.3 | 6.67 | 21 |
| 68 | MEPI | 3 | mannitol | 35 | MAP | 0.3 | 6.67 | 27 |
| 69 | none | 0 | mannitol | 35 | MAP | 0.7 | n/a | 41 |
| 70 | LIDO | 3 | mannitol | 35 | MAP | 0.7 | 6.67 | 24 |
| 71 | MEPI | 3 | mannitol | 35 | MAP | 0.7 | 6.67 | 31 |
| 63 | none | 0 | mannitol | 35 | SOS | 1 | n/a | 40 |
| 64 | LIDO | 3 | mannitol | 35 | SOS | 1 | 6.67 | 27 |
| 65 | MEPI | 3 | mannitol | 35 | SOS | 1 | 6.67 | 31 |

The rheological properties during heat sterilization of compositions comprising hyaluronic acid with a weight-average molecular mass of 3×10⁶ Da at a concentration of 20 mg/g with a degree of crosslinking X=0.12 are less impaired in the presence of mepivacaine, relative to the reference composition.

### Example 9-e

Example 9-e consists of a compilation of the tests cited above relating to interpenetrating hyaluronic acid prepared according to patent application WO 2009/071 697 in the name of Vivacy.

All the calculations of % G′ are performed taking as reference the composition comprising neither polyol nor local anesthetic nor additional compound.

TABLE 23

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 72 | none | 0 | none | 0 | none | 0 | n/a | 0 |
| 75 | none | 0 | mannitol | 35 | none | 0 | n/a | 54 |
| 73 | LIDO | 3 | none | 0 | none | 0 | 6.67 | −9 |
| 74 | MEPI | 3 | none | 0 | none | 0 | 6.67 | 8 |
| 76 | LIDO | 3 | mannitol | 35 | none | 0 | 6.67 | −4 |
| 77 | MEPI | 3 | mannitol | 35 | none | 0 | 6.67 | 12 |

The rheological properties during heat sterilization of compositions comprising interpenetrating hyaluronic acid prepared according to patent application WO 2009/071 697 in the name of Vivacy are less impaired in the presence of mepivacaine, relative to the reference composition.

### Example 9-f

Example 9-f consists of a compilation of the tests cited above relating to co-crosslinked hyaluronic acid prepared according to patent application WO 86/00079 in the name of Allergan.

All the calculations of % G′ are performed taking as reference the composition comprising neither polyol nor local anesthetic nor additional compound.

TABLE 24

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 78 | none | 0 | none | 0 | none | 0 | n/a | 0 |
| 81 | none | 0 | mannitol | 35 | none | 0 | n/a | 24 |
| 79 | LIDO | 3 | none | 0 | none | 0 | 6.67 | 22 |
| 80 | MEPI | 3 | none | 0 | none | 0 | 6.67 | 26 |
| 82 | LIDO | 3 | mannitol | 35 | none | 0 | 6.67 | −1 |
| 83 | MEPI | 3 | mannitol | 35 | none | 0 | 6.67 | 7 |

The rheological properties during heat sterilization of compositions comprising co-crosslinked hyaluronic acid prepared according to patent application WO 86/00079 in the name of Allergan are less impaired in the presence of mepivacaine, relative to the reference composition.

### Example 10

Example 10 illustrates that the influence of adding various anesthetics on the impairment during heat sterilization of the rheological properties of hyaluronic acid gels is verified over a ratio range [HA]/[MEPI]≧0.1 and at the very least between 0.4 and 2500.

TABLE 25

| | LA | | Aox | | AC | | [HA]/[MEPI] | |
|---|---|---|---|---|---|---|---|---|
| | | [LA] | | [Aox] | | [AC] | or | |
| No. | nature | (mg/g) | nature | (mg/g) | nature | (mg/g) | [HA]/[LA] | % G′ |
| 84 | MEPI | 0.01 | none | 0 | none | | 2500 | 0 |
| 85 | LIDO | 0.01 | none | 0 | none | 0 | 2500 | −7 |
| 86 | ARTI | 0.01 | none | 0 | none | 0 | 2500 | −14 |
| 87 | PRILO | 0.01 | none | 0 | none | 0 | 2500 | −6 |
| 88 | MEPI | 25 | none | 0 | none | | 0.4 | 0 |
| 89 | LIDO | 25 | none | 0 | none | 0 | 0.4 | −34 |
| 90 | ARTI | 25 | none | 0 | none | 0 | 0.40 | −49 |
| 91 | PRILO | 25 | none | 0 | none | 0 | 0.40 | −52 |

1. A sterilized aqueous composition, at a pH close to physiological pH, comprising:
at least one hyaluronic acid or a salt thereof;
mepivacaine or pharmaceutically acceptable salt thereof; and
at least one antioxidant that is a polyol,
wherein the mass ratio between the concentration of hyaluronic acid or salt thereof [HA] and the concentration of mepivacaine or salt thereof [MEPI]:[HA]/[MEPI] is greater than or equal to 0.1 ([HA]/[MEPI]≧0.1).

2. The sterilized aqueous composition as claimed in claim 1, wherein [HA]/[MEPI] is between 0.1 and 50 (0.1≦[HA]/[MEPI]≦50).

3. The sterilized aqueous composition as claimed in claim 1, wherein the concentration of mepivacaine or salt thereof [MEPI] is between 0.01 mg/g and 50 mg/g of total weight of said composition.

4. The sterilized aqueous composition as claimed in claim 1, wherein mepivacaine or salt thereof is selected from the group consisting of racemic mepivacaine hydrochloride, (R)-mepivacaine hydrochloride, (S)-mepivacaine hydrochloride, racemic mepivacaine, (R)-mepivacaine, (S)-mepivacaine, and pharmaceutically acceptable salts thereof.

5. The sterilized aqueous composition as claimed in claim 1, wherein the concentration of hyaluronic acid or salt thereof [HA] is between 2 mg/g and 50 mg/g of total weight of said composition.

6. The sterilized aqueous composition as claimed in claim 1, comprising at least one non-crosslinked hyaluronic acid or a salt thereof, alone or as a mixture.

7. The sterilized aqueous composition as claimed in claim 1, comprising at least one crosslinked hyaluronic acid or a salt thereof, alone or as a mixture.

8. The sterilized aqueous composition as claimed in claim 1, wherein the polyol is selected from the group consisting of glycerol, sorbitol, propylene glycol, xylitol, mannitol, erythritol, maltitol, lactitol, and mixtures thereof.

9. The sterilized aqueous composition as claimed in claim 1, further comprising at least one additional compound.

10. A process of manufacturing the sterilized aqueous composition as claimed in claim 1, comprising:
a step of hydration in a buffer solution at a pH close to physiological pH of fibers of at least one hyaluronic acid or a salt thereof, alone or as a mixture, to obtain a hydrogel;
a step of incorporating mepivacaine or pharmaceutically acceptable salt thereof as an aqueous solution in the hydrogel;
a step of incorporating at least one antioxidant that is a polyol in the hydrogel;
a homogenization step; and
a sterilization step.

11. The process as claimed in claim 10, further comprising at least one crosslinking step.

12. A method for filling wrinkles, for correcting skin defects, or for cheekbone, chin, or lips volumizing comprising using the sterilized aqueous composition as claimed in claim 1 to fill wrinkles, correct skin defects, or volumize a cheekbone, chin, or lips.

13. A method for replacement or supplement of deficient synovial fluid of a joint comprising injecting the sterilized aqueous composition as claimed in claim 1 into the joint.

14. A kit comprising the sterilized aqueous composition as claimed in claim 1 packaged in a syringe and sterilized after packaging.

15. The sterilized aqueous composition as claimed in claim 1, wherein said composition is formulated for use as a replacement or supplement for deficient synovial fluid.
